(19) United States
(12) Patent Application Publication
(10) Pub. No.: US 2004/0072184 A1
(43) Pub. Date: Apr. 15, 2004

(54) Cancer associated protein kinases and their uses

(76) Inventors: Thillainathan Yoganathan, Richmond (CA); Allen D. Delaney, Vancouver (CA)

Correspondence Address: BOZICEVIC, FIELD & FRANCIS LLP, 200 MIDDLEFIELD RD, SUITE 200, MENLO PARK, CA, 94025, US

(21) Appl. No.: 10/380,235

(22) PCT Filed: Sep. 20, 2001
(86) PCT No.: PCT/IB01/02237

(51) Int. Cl. C12Q 1/68; G01N 33/574

(52) U.S. Cl. 435/6; 435/7.23

(57) ABSTRACT

Detection of expression of the provided protein kinase in cancers is useful as a diagnostic, for determining the effectiveness of drugs, and determining patient prognosis. The encoded polypeptides further provides a target for screening pharmaceutical agents effective in inhibiting the growth or metastasis of tumor cells

An accumulation of genetic changes underlies the development and progression of cancer, resulting in cells that differ from normal cells in their behavior, biochemistry, genetics, and microscopic appearance. Mutations in DNA that cause changes in the expression level of key proteins, or in the biological activity of proteins, are thought to be at the heart of cancer. For example, cancer can be triggered in part when genes that play a critical role in the regulation of cell division undergo mutations that lead to their over-expression. “Oncogenes” are involved in the dysregulation of growth that occurs in cancers.

Oncogene activity may involve protein kinases, enzymes that help regulate many cellular activities, particularly signaling from the cell membrane to the nucleus to initiate the cell's entrance into the cell cycle and to control other functions.

Oncogenes may be tumor susceptibility genes, which are typically up-regulated in tumor cells, or may be tumor suppressor genes, which are down-regulated or absent in tumor cells. Malignancies can arise when a tumor suppressor is lost and/or an oncogene is inappropriately activated. When such mutations occur in somatic cells, they result in the growth of sporadic tumors.

Hundreds of genes have been implicated in cancer, but in most cases relationships between these genes and their effects are poorly understood. Using massively parallel gene expression analysis, scientists can now begin to connect these genes into related pathways.

Phosphorylation is important in signal transduction mediated by receptors via extracellular biological signals such as growth factors or hormones. For example, many oncogenes are protein kinases, i.e. enzymes that catalyze protein phosphorylation reactions or are specifically regulated by phosphorylation. In addition, a kinase can have its activity regulated by one or more distinct protein kinases, resulting in specific signaling cascades.

Cloning procedures aided by homology searches of EST databases have accelerated the pace of discovery of new genes, but EST database searching remains an involved and onerous task. More than 1.6 million human EST sequences have been deposited in public databases, making it difficult to identify ESTs that represent new genes. Compounding the problems of scale are difficulties in detection associated with a high sequencing error rate and low sequence similarity between distant homologues.

Despite a long-felt need to understand and discover methods for regulating cells involved in various disease states, the complexity of signal transduction pathways has been a barrier to the development of products and processes for such regulation. Accordingly, there is a need in the art for improved methods for detecting and modulating the activity of such genes, and for treating diseases associated with the cancer and signal transduction pathway.

Relevant Literature

The use of genomic sequence in data mining for signaling proteins is discussed in Schultz et al. (2000) Nature Genetics 25:201. The MAPK protein family has been reviewed, for example by Meskiene I, and Hirt, H. (2000) Plant Mol Biol 42(6):791-806. MAP3K has been discussed, for example, by Ing, Y. L. et al. (1994) Oncogene. 9: 1745-1750:and also by Courseaux A. e.al. (1996) Genomics, 37:354-365 Serine/threonine protein kinases have been reviewed, for example, by Cross TG, et al.(2000) Exp Cell Res. Apr 10;256(1):34-41.

### SUMMARY OF THE INVENTION

The genetic sequences provided herein as SEQ ID NOS:1, 3, 5, 7, 9, 11 and 13 encode protein kineses that are herein shown to be over-expressed In cancer cells. Detection of expression in cancer cells is useful as a diagnostic; for determining the effectiveness and mechanism of action of therapeutic drug candidates, and for determining patient prognosis. These sequences further provides a target for screening pharmaceutical agents effective in inhibiting the growth or metastasis of tumor cells. In one embodiment of the invention, a complete nucleotide sequence of the human cDNA corresponding to the cancer associated protein kinase Is provided.

### BRIEF DESCRIPTION OF THE DRAWINGS

FIG. 1 Is a graph depicting the proliferation of Cos7 cells that were transfected with Increasing concentrations of CaMK-X1 or vector plasmids in the presence of KCl.

FIG. 2 is a graph depicting phosphorylation of CREBtide and Syntide 2 in vitro by CamKX1.

FIG. 3 is a graph depicting activity of transcription factors in the presence of SGK2. AP1 and NF-κB activity was measured in HEK293 cells and in HEK293 cells stably transfected with SGK2.

FIG. 4 is a graph depicting the activation of SGK2 (K 25 plasmid) by PDK1.

FIG. 5 depicts the sequences of several DMPK isoforms.

### DESCRIPTION OF THE SPECIFIC EMBODIMENTS

SEQ ID NOS:1, 3, 5, 7, 9, 11 and 13 encode protein kinases that are shown to be over-expressed in cancer cells. The encoded cancer associated protein kinases of the invention provide targets for drug screening or altering expression levels, and for determining other molecular targets in kinase signal transduction pathways involved in transformation and growth of tumor cells. Detection of over-expression in cancers provides a useful diagnostic for predicting patient prognosis and probability of drug effectiveness.

### Protein Kinases

Mitogen Activated Protein Kinases. The human gene sequence encoding MAP3K11, Is provided as SEQ ID NO:1, and the encoded polypeptide product is provided as SEQ ID NO: 2. Dot blot analysis of probes prepared from mRNA of tumors showed that expression of MAP3K11 is consistently up-regulated in clinical samples of human tumors.

Many of the transduction pathways in mammalian cells that involve the sequential activation of a series of signaling proteins linking the cell surface with nuclear targets are mediated by mitogen-activated protein kinases (MAPKs) (also called extracellular signal-regulated kinases or ERKs). In mammalian cells, three parallel MAPK pathways have been described. Generally, MAPKs are rapidly activated in response to ligand binding by both growth factor receptors that are tyrosine kinases (such as the EGF receptor) and receptors that are coupled to G proteins. Phosphorylation of tyrosine residues leads to generation of docking sites for SH2 (Src homology 2) and PTB (phosphotyrosine binding) domains of adaptor proteins. (see Lemmon et at. (1994) Trends Bioch m Sci 19:459-63; and Pawson et al. (1997) Science 278:2075-80.

Mitogen-activated protein (MAP) kinases include extracellular signal-regulated protein kinase (ERK), c-Jun amino-terminal kinase (JNK), and p38 subgroups. These MAP kinase Isoforms are activated by dual phosphorylation on threonine and tyrosine (Derijard et al. (1995) Science 267(5198):682-5). MAP3K11 is an isoform that has been described by Ing et al. (1994) Oncogen 9:1745-1750. It has been mapped via fluorescence in situ hybridization to 11q13.1-q13.3 (Courseaux et. al. (1996) Genomics 37:354-365). MAP3K also shares homology, including an unusual leucine zipper-basic motif, with a family of protein kinases known as mixed lineage protein kinases.

Ing et. al. (supra.) found that MAP3K contains an SH3 domain and has a long carboxy-terminal tail that exhibits proline rich motifs similar to known SH3 binding sites. SH3 domains play the role of a protein switch, which is turned on by a number of receptor-mediated signals to which it responds by changes in kinase activity and by changes in intracellular localization. It acts as part of an adapter molecule and recruits downstream proteins in a signaling pathway.

Calmodulin Kinase. The human gene sequence encoding CaMK-X1, which maps to chromosome 1q32.1-32.3, is provided as SEQ ID NO:3, and the encoded polypeptide product is provided as SEQ ID NO: 4. The open reading frame of the sequence is Indicated in the seqlist of SEQ ID NO:3, and starts at position 70. Dot blot analysis of probes prepared from mRNA of tumors showed that expression of CaMK-X11 is consistently up-regulated in human tumor tissue.

Many of the intracellular physiological activities in mammalian cells that Involve Ca⁺⁺ as a second messenger are mediated by calmodulin (CAM). This ubiquitous Ca⁺⁺-binding protein has an ability to activate a variety of enzymes in a Ca⁺⁺-dependent manner. Among these enzymes are Ca⁺⁺ and calmodulin-dependent cyclic-nucleotide phosphodiesterase (CaM-PDE) and the calmodulin-dependent kinases. Many of the CaM-kinases are activated by phosphorylation in addition to binding to CaM. The kinase may autophosphorylate, or be phosphorylated by another kinase as part of a “kinase cascade”.

Each member of the CaM-kinase cascade has a catalytic domain adjacent to a regulatory region that contains an overlapping auto-inhibitory domain (AID) and the CaM-binding domain (CBD). An interaction between the AID and the catalytic domain maintains the kinase in an inactive conformation by preventing binding of protein substrate as well as Mg⁺⁺-ATP. Binding of Ca⁺⁺-CaM to the CBD alters the conformation of the overlapping AID such that it no longer interferes with substrate binding; the kinase is therefore active. As in the cases of other protein kinases, CaMKI has a catalytic cleft between its upper and lower lobes, which are responsible for binding Mg⁺⁺-ATP and protein substrates, respectively. At the base of their catalytic clefts, many protein kinases, Including CaMKI and CaMKIV, have an activation loop containing a threonine residue whose phosphorylation strongly augments kinase activity.

Serum and Glucocorticoid-induced. Protein Kinases (SGk). The human gene sequence encoding SGK2-α is provided as SEQ ID NO:5, and the encoded polypeptide product is provided as SEQ ID NO:6. Dot blot analysis of probes prepared from mRNA of tumors showed that expression of SGK2-α is consistently up-regulated in human tumor tissue.

SGKs actively shuttle between the nucleus and the cytoplasm in synchrony with the cell cycle. SGK was originally identified as a glucocorticoid and osmotic stress-responsive gene; two related isoforms have been termed SGK2 and SGK3. In addition, there are two splice variants of SGK2; specifically, SGK2α and SGK2β. SGK2α encodes a protein of 367 residues with a calculated molecular mass of 41.1 kDa. Although SGK 1, 2, and 3 share a high degree of sequence similarity, the mechanisms that regulate the level and activity of SGK2 and SGK3 differ significantly from those that regulate SGK1. SGK2 has a peptide specificity similar to that of protein kinase B, preferentially phosphorylating Ser and Thr residues that lie in Arg-Xaa-Arg-Xaa-Xaa-Ser/Thr motifs.

The data provided herein demonstrate that SGK2α is activated by protein dependent kinase 1. CDK1 is a catalytic subunit of a protein kinase complex, called the M-phase promoting factor, that induces entry into mitosis and is universal among eukaryotes. Lee et at. (1988) Nature 333: 676-679 describe the regulated expression and phosphorylation of CDK1 in human and murine in vitro systems. Serum stimulation of human and mouse fibroblasts results in a markedincrease in CDK1 transcription. Both the yeast and mammalian systems are regulated by phosphorylaton of the gene product. In HeLa cells, CDK1 is the most abundant phosphotyrosine-containing protein and its phosphotyrosine content is subject to cell-cycle regulation (Draetta et al. (1988) Nature 336: 738-744). One site of CDK1 tyrosine phosphorylation in vivo is selectively phosphorylated in vitro by a product of the SRC gene. Taxol activates CDK1 kinase in MDA-MB435 breast cancer cells, leading to cell cycle arrest at the G2/M phase and, subsequently, apoptosis. Chemical Inhibitors of CDK1 block taxol-induced apoptosis in these cells (Yu et al. (1998) Molec. Cell 2:581-591). Interference in this pathway is of interest in the development of therapeutic agents that affect cell cycle arrest and apoptosis.

G Protein coupled Receptor Kinase. The human gene sequence encoding GRK5 is provided as SEQ ID NO:7, and the encoded polypeptide product is provided as SEQ ID NO:8. Dot blot analysis of probes prepared from mRNA of tumors showed that expression of GRK5 is consistently up-regulated in clinical samples of human tumors.

GRKs are a family of serine/threonine kinases that induce receptor desensitization by the phosphorylation of agonist-occupied or -activated receptors. GRKs transduce the binding of extracellular ligands into intracellular signaling events. To date, seven members of the GRK family have been identified. Common features of these kinases include a centrally localized catalytic domain of approximately 240 amino acids, which shares significant sequence identity between family members, an N-terminal domain of 161-197 amino acids, and a variable length C-terminal domain. All of the GRKs can directly interact with phospholipids either via covalent modifications such as farnesylation, palmitoylation, or via lipid binding domains such as the pleckstrin homology domain, or a polybasic domain.

GRK5 is a protein of approximately 67.7 kDa (see Kunapali and Benovic (1993) P.N.A.S. 90:5588-5592) and was identified by its homology with other members of the GRK family. It is expressed in a number of different tissues, including heart, placenta and lung. Autophosphorylation of GRK5 appears to activate the kinase (Pronin and Benovic (1997) P.N.A.S. 272:3806-3812). GRK5 is also phosphorylated by PKC, where the major sites of PKC phosphorylation are localized within the C-terminal 26 amino acids. PKC phosphorylation significantly inhibits GRK5 activity.

Myotonic dystrophy protein kinase. The human gene sequence encoding DM-PK, is provided as SEQ ID NO:9, and the encoded polypeptide product is provided as SEQ ID NO: 10. The sequence of additional isoforms is provided as SEQ ID NO:38 and SEQ ID NO:39. Dot blot analysis of probes prepared from mRNA of tumors showed that expression of DM-PK is consistently up-regulated in clinical samples of human tumors.

Human myotonic dystrophy protein kinase (DM-PK) is a member of a class of multidomain protein kinases that regulate cell size and shape in a variety of organisms (see Brook et al. (1992) Cell 68:799-808; and Fu et al. (1992) Science 255:1256-1258). DM-PK exhibits a novel catalytic activity similar to, but distinct from, related protein kinases such as protein kinase C and A, and the Rho kinases. Little is currently known about the general properties of DM-PK including domain function, substrate specificity, and potential mechanisms of regulation. Two forms of the kinase are expressed in muscle, where the larger form (the primary translation product) is proteolytically cleaved near the carboxy terminus to generate the smaller. Inhibitory activity of the full-length kinase has been mapped to a pseudosubstrate autoinhibitory domain at the extreme carboxy terminus of DM-PK (see Bush et al. (2000) Biochemistry 39:8480-90).

Shaw et al. (1993) Genomics 18:673-9 demonstrated that the DM-PK gene contains 15 exons distributed over about 13 kb of genomic DNA. It encodes a protein of 624 amino acids with an N-terminal domain highly homologous to cAMP-dependent serine-threonine protein kinases, an intermediate domain with a high alpha-helical content and weak similarity to various filamentous proteins, and a hydrophobic C-terminal segment. A CTG repeat is located in the 3′ untranslated region of DM-PK mRNA. The unstable CTG motif is found uniquely in humans, although the flanking nucleotides are also present in mouse. The involvement of a protein kinase in myotonic dystrophy is consistent with the pivotal role of such enzymes in a wide range of biochemical and cellular pathways. The autosomal dominant nature of the disease Is due to a dosage deficiency.

Protein Kinase D2. The human gene sequence encoding PKD2 Is provided as SEQ ID NO:11, and the encoded polypeptide product is provided as SEQ ID NO:12. Dot blot analysis of probes prepared from mRNA of tumors showed that expression of PKD2 is consistently up-regulated in clinical samples of human tumors.

PKD2 is a human serine threonine protein kinase gene (Genbank accession number NM_{—}016457; Sturany et al. (2001) J. Biol. Chem. 276:3310-3318). The prot In sequence contains two cysteine-rich motifs at the N terminus, a pleckstrin homology domain, and a catalytic domain containing all the characteristic sequence motifs of serine protein kinases. It exhibits the strongest homology to the serine threonine protein kinases PKD/PKCμ and PKC, particularly in the duplex zinc finger-like cysteine-rich motif, in the pleckstrin homology domain and in the protein kinase domain. The mRNA of PKD2 is widely expressed in human and murine tissues. It encodes a protein with a molecular mass of 105 kDa in SDS-polyacrylamide gel electrophoresis, which is expressed in various human cell lines, including HL60 cells, which do not express PKCμ. In vivo phorbol ester binding studies demonstrated a concentration-dependent binding of [³H]phorbol 12,13dibutyrate to PKD2. The addition of phorbol 12,13-dibutyrate in the presence of dioleoylphosphatidylserine stimulated the autophosphorylation of PKD2 in a synergistic fashion. Phorbol esters also stimulated autophosphorylation of PKD2 in intact cells. Phosphorylation of Ser876 of PKD2 correlated with the activation status of the kinase.

### Diagnostic Methods

Determination of the presence of any one of SEQ ID NOS:1, 3, 5, 7, 9, 11 and 13 is used in the diagnosis, typing and staging of tumors. Detection of the presence of the sequence is performed by the use of a specific binding pair member to quantitate the specific protein, DNA or RNA present in a patient sample. Generally the sample will be a biopsy or other cell sample from the tumor. Where the tumor has metastasized, blood samples may be analyzed.

### Specific Binding Members

In a typical assay, a tissue sample, e.g. biopsy, blood sample, etc. is assayed for the presence of a cancer associated kinase corresponding to SEQ ID NOS:1, 3, 5, 7, 9, 11 or 13 specific sequences by combining the sample with a SEQ ID NOS:1, 3, 5, 7, 9, 11 and 13 specific binding member, and detecting directly or indirectly the presence of the complex formed between the two members. The term “specific binding member” as used herein refers to a member of a specific binding pair, i.e. two molecules where one of the molecules through chemical or physical means specifically binds to the other molecule. One of the molecules will be a nucleic acid corresponding to SEQ ID NOS:1, 3, 5, 7, 9, 11 and 13 or a polypeptide encoded by the nucleic acid, which can include any protein substantially similar to the amino acid sequence provided in SEQ ID NOs:2, 4, 6, 8, 10, 12, 14, 38 or 39 or a fragment thereof; or any nucleic acid substantially similar to the nucleotide sequence provided in SEQ ID NOS:1, 3, 5, 7, 9, 11 and 13, or a fragment thereof. The complementary members of a specific binding pair are sometimes referred to as a ligand and receptor.

Binding pairs of interest include antigen and antibody specific binding pairs, peptide-MHC antigen and T cell receptor pairs; complementary nucleotide sequences (including nucleic acid sequences used as probes and capture agents in DNA hybridizaton assays); kinase protein and substrate pairs; autologous monoclonal antibodies, and the like. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present.

Nucleic acid sequences. In another embodiment of the invention, nucleic acids are used as a specific binding member. Sequences for detection are complementary to a one of the provided cancer associated kinase corresponding to SEQ ID NOS:1, 3, 5, 7, 9, 11 or 13. The nucleic acids of the invention include nucleic acids having a high degree of sequence similarity or sequence identity to one of SEQ ID NOS:1, 3, 5, 7, 9, 11 and 13. Sequence identity can be determined by hybridization under stringent conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided nucleic acid sequence, e.g. allelic variants, genetically altered versions of the gene, etc., bind to SEQ ID NOS:1, 3, 5, 7, 9, 11 or 13 under stringent hybridization conditions.

The nucleic acids can be cDNAs or genomic DNAs, as well as fragments thereof. The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide of the invention.

A genomic sequence of interest comprises the nucleic acid present between the initiation codon and the stop codon, as defined in the listed sequences, including all of the introns that are normally present in a native chromosome. It can further Include the 3′ and 5′ untranslated regions found in the mature mRNA. It can further include specific transcription and translational regulatory sequences, such as promoters, enhancers, etc., including about 1 kb, but possibly more, of flanking genomic DNA at either the 5′ or 3′ end of the transcribed region. The genomic DNA flanking the coding region, either 3′ or 5′, or internal regulatory sequences as sometimes found in introns, contains sequences required for proper tissue, stage-specific, or disease-state specific expression, and are useful for investigating the up-regulation of expression In tumor cells.

Probes specific to the nucleic acid of the invention can be generated using the nucleic acid sequence disclosed in SEQ ID NOS:1, 3, 5, 7, 9, 11 or 13. The probes are preferably at least about 18 nt, 25 nt, 50 nt or more of the corresponding contiguous sequence of SEQ ID NOS:1, 3, 5, 7, 9, 11 or 13, and are usually less than about 2, 1, or 0.5 kb in length. Preferably, probes are designed based on a contiguous sequence that remains unmasked following application of a masking program for masking low complexity, e.g. BLASTX Double or single stranded fragments can be obtained from the DNA sequence by chemically synthesizing oligonucleotides in accordance with conventional methods, by restriction enzyme digestion, by PCR amplification, etc. The probes can be labeled, for example, with a radioactive, biotinylated, or fluorescent tag.

The nucleic acids of the subject invention are isolated and obtained in substantial purity, generally as other than an intact chromosome. Usually, the nucleic adds, either as DNA or RNA, will be obtained substantially free of other naturally-occurring nucleic add sequences, generally being at least about 50%, usually at least about 90% pure and are typically “recombinant,” .g., flanked by one or more nucleotides with which it is not normally associated on a naturally occurring chromosome.

The nucleic acids of the invention can be provided as a linear molecule or within a circular molecule, and can be provided within autonomously replicating molecules (vectors) or within molecules without replication sequences. Expression of the nucleic acids can be regulated by their own or by other regulatory sequences known in the art. The nucleic acids of the invention can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

For use in amplification reactions, such as PCR, a pair of primers will be used. The exact composition of the primer sequences is not critical to the invention, but for most applications the primers will hybridize to the subject sequence under stringent conditions, as known in the art. It is preferable to choose a pair of primers that will generate an amplification product of at least about 50 nt, preferably at least about 100 nt. Algorithms for the selection of primer sequences are generally known, and are available in commercial software packages. Amplification primers hybridize to complementary strands of DNA, and will prime towards each other. For hybridization probes, it may be desirable to use nucleic acid analogs, in order to improve the stability and binding affinity. The term “nucleic acid” shall be understood to encompass such analogs.

Antibodies. The polypeptides of the invention may be used for the production of antibodies, where short fragments provide for antibodies specific for the particular polypeptide, and larger fragments or the entire protein allow for the production of antibodies over the surface of the polypeptide. As used herein, the term “antibodies” includes antibodies of any isotype, fragments of antibodies which retain specific binding to antigen, including, but not limited to, Fab, Fv, scfv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a green fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

“Antibody specificity”, in the context of antibody-antigen interactions, is a term well understood in the art, and indicates that a given antibody binds to a given antig n, wherein the binding can be inhibited by that antigen or an epitope thereof which is recognized by the antibody, and does not substantially bind to unrelated antigens. Methods of determining specific antibody binding are well known to those skilled in the art, and can be used to determine the specificity of antibodies of the invention for a polypeptide, particularly a human polypeptide corresponding to SEQ ID NOS:2, 4, 6, 8, 10 or 12.

Antibodies are prepared in accordance with conventional ways, where the expressed polypeptide or protein is used as an immunogen, by itself or conjugated to known Immunogenic carriers, e.g. KLH, pre-S HBsAg, other viral or eukaryotic proteins, or the like. Various adjuvants may be employed, with a series of injections, as appropriate. For monoclonal antibodies, after one or more booster injections, the spleen is isolated, the lymphocytes immortalized by cell fusion, and then screened for high affinity antibody binding. The immortalized cells, i.e. hybridomas, producing the desired antibodies may then be expanded. For further description, see Monoclonal Antibodies: A Laboratory Manual, Harlow and Lane eds., Cold Spring Harbor Laboratories, Cold Spring Harbor, N.Y., 1988. If desired, the mRNA encoding the heavy and light chains may be isolated and mutagenized by cloning in E. coli, and the heavy and light chains mixed to further enhance the affinity of the antibody. Alternatives to in vivo immunization as a method of raising antibodies include binding to phage display libraries, usually in conjunction with in vitro affinity maturation.

### Methods for Quantitation of Nucleic Acids

Nucleic acid reagents derived from the sequence of SEQ ID NOS:1, 3, 5, 7, 9, 11 or 13 are used to screen patient samples, e.g. biopsy-derived tumors, inflammatory samples such as arthritic synovium, etc:, for amplified DNA in the cell, or increased expression of the corresponding mRNA or protein. DNA-based reagents are also designed for evaluation of chromosomal loci implicated in certain diseases e.g. for use in loss-of-heterozygosity (LOH studies, or design of primers based on coding sequences.

The polynucleotides of the invention can be used to detect differences in expression levels between two cell, e.g., as a method to identify abnormal or diseased tissue in a human. The tissue suspected of being abnormal or diseased can be derived from a different tissue type of the human, but preferably it is derived from the same tissue type; for example, an intestinal polyp or other abnormal growth should be compared with normal intestinal tissue. The normal tissue can be the same tissue as that of the test sample, or any normal tissue of the patient, especially those that express the polynucleotide-related gene of interest (e.g., brain, thymus, tests, heart, prostate, placenta, spleen, small intestine, skeletal muscle, pancreas, and the mucosal lining of the colon, etc.). A difference between the polynucleotide-related gene, mRNA, or protein in the two tissues which are compared, for example, in molecular weight, amino acid or nucleotide sequence, or relative abundance, indicates a change in the gene, or a gene which regulates It, In the tissue of the human that was suspected of being diseased.

The subject nucleic acid and/or polypeptide compositions may be used to analyze a patient sample for the presence of polymorphisms associated with a disease state. Biochemical studies may be performed to determine whether a sequence polymorphism in a coding region or control regions is associated with disease, particularly cancers and other growth abnormalities. Diseases of interest may also include other hyperproliferative disorders. Disease associated polymorphisms may include deletion or truncation of the gene, mutations that alter expression level, that affect the binding activity of the protein, the kinase activity domain, etc.

Changes in the promoter or enhancer sequence that may affect expression levels of can be compared to expression levels of the normal allele by various methods known n the art Methods for determining promoter or enhancer strength include quantitation of the expressed natural protein insertion of the variant control element into a vector with a reporter gene such as beta-galactosidase, luciferase, chloramphenicol acetyltransferase, etc. that provides for convenient quantitation; and the like.

A number of methods are available for analyzing nucleic acids for the presence of a specific sequence, e.g. upregulated expression. Cells that express SEQ ID NOS:1, 3, 5, 7, 9, 11 or 13 may be used as a source of mRNA, which may be assayed directly or reverse transcribed into cDNA for analysis. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of techniques may be found in Sambrook, et al. Molecular Cloning: A Laboratory Manual, CSH Press 1989, pp.14.2-14.33.

A detectable label may be included in an amplification reaction. Suitable labels include fluorochromes, e.g. fluorescein isothiocyanate (FITC), rhodamine, Texas Red, phycoerythrin, allophycocyanin,6-carboxyfluorescein(6-FAM),2,7-dimethoxy-4,5-dichloro-6-carboxyfluorescein (JOE), 6-carboxy-X-rhodamine (ROX), 6-carboxy-2,4,7,4,7-hexachlorofluorescein (HEX) 5-carboxyfluorescein (5-FAM) or N,N,N,N-tetramethyl-6-carboxyrhodamine (TAMRA), radioactive labels, e.g. ³²p ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis(DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

Arrays provide a high throughput technique that can assay a large number of polynucleotides in a sample. In one aspect of the invention, an array is constructed comprising one or more of SEQ ID NOS:1, 3, 5, 7, 9, 11 and 13, preferably comprising all of these sequences, which array may further comprise other sequences known to be up or down-regulated in tumor cells. This technology can be used as a to I to test for differential expression.

A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions. Samples of nucleic acids can be detectably-labeled (e.g. using radioactive or fluorescent labels) and then hybridized to the probes. Double stranded nucleic acids, comprising the labeled sample polynucleotides bound to probe nucleic acids, can be detected once the unbound portion of the sample is washed away. Alternatively, the nucleic acids of the test sample can be immobilized on the array, and the probes detectably labeled;

Techniques for constructing arrays and methods of using these arrays are described in, for example, Schena et al. (1996) Proc Natl Acad Sci USA. 93(20):10614-9; Schena et at. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/62357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

Arrays can be used to, for example, examine differential expression of genes and can be used to determine gene function. For example, arrays can be used to detect differential expression of SEQ ID NOS:1, 3, 5, 7, 9, 11 or 13, where expression is compared between a test cell and control cell (e.g., cancer cells and normal cells). High expression of a particular message in a cancer cell, which is not observed in a corresponding normal cell, indicates a cancer specific gene product. Exemplary uses of arrays are further described in, for example, Pappalarado. et al. (1998) Sem. Radiation Oncol. 8:217; and Ramsay. (1998) Nature Biotechnol. 16.40. Furthermore, many variations on methods of detection using arrays are well within the skill In the art and within the scope of the present invention. For example, rather than immobilizing the probe to a solid support, the test sample can be immobilized on a solid support which is then contacted with the probe.

### Polypeptide Analysis

Screening for expression of the subject sequences may be based on the functional or antigenic characteristics of the protein. Protein truncation assays are useful in detecting deletions that may affect the biological activity of the protein. Various immunoassays designed to detect polymorphisms in proteins encoded by SEQ ID NOS:1, 3, 5, 7, 9, 11 or 13 may be used in screening. Where many diverse genetic mutations lead to a particular disease phenotype, functional protein assays have proven to be effective screening tools. The activity of the encoded protein in kinase assays, etc., may be determined by comparison with the wild-type protein.

A sample is taken from a patient with cancer. Samples, as used herein, include biological fluids such as blood; organ or tissue culture derived fluids; etc. Biopsy samples or other sources of carcinoma cells are of particular interest, e.g. tumor biopsy, etc. Also included in th term are derivatives and fractions of such cells and fluids. The number of cells in a sample will generally be at least about 10³, usually at least 10⁴, and may be about 10⁵ or more. The cells may be dissociated, in the case of solid tissues, or tissue sections may be analyzed. Alternatively a lysate of the cells may be prepared.

Detection may utilize staining of cells or histological sections, performed in accordance with conventional methods. The antibodies or other specific binding members of interest are added to the cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

An alternative method for diagnosis depends on the in vitro detection of binding between antibodies and the cancer associated kinase corresponding to SEQ ID NOS:1, 3, 5, 7, 9, 11 or 13 in a lysate. Measuring the concentration of the target protein in a sample or fraction thereof may be accomplished by a variety of specific assays. A conventional sandwich type assay may be used. For example, a sandwich assay may first attach specific antibodies to an insoluble surface or support. The particular manner of binding is not crucial so long as it is compatible with the reagents and overall methods of the invention. They may be bound to the plates covalently or non-covalently, preferably non-covalently.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Patient sample lysates are then added to separately assayable supports (for example, separate wells of a microtiter plate) containing antibodies. Preferably, a series of standards, containing known concentrations of the test protein is assayed in parallel with the samples or aliquots thereof to serve as controls. Preferably, each sample and standard will be added to multiple wells so that mean values can be obtained for each. The incubation time should be sufficient for binding, generally, from about 0.1 to 3 hr is sufficient. After incubation, the insoluble support is generally washed of non-bound components. Generally, a dilute non-ionic detergent medium at an appropriate pH, generally 7-8, is used as a wash medium. From one to six washes may be employed, with sufficient volume to thoroughly wash non-specifically bound proteins present in the sample.

After washing, a solution containing a second antibody is applied. The antibody will bind to one of the proteins encoded by SEQ ID NOS:1, 3, 5, 7, 9, 11 or 13 with sufficient specificity such that it can be distinguished from other components present The second antibodies may be labeled to facilitate direct, or indirect quantification of binding. Examples of labels that permit direct measurement of second receptor binding include radiolabels, such as ³H or ¹²⁵I, fluorescers, dyes, beads, chemiluminescers, colloidal particles, and the like. Examples of labels that permit indirect measurement of binding include enzymes where the substrate may provide for a colored or fluorescent product. In a preferred embodiment, the antibodies are labeled with a covalently bound enzyme capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. The incubation time should be sufficient for the labeled ligand to bind available molecules. Generally, from about 0.1 to 3 hr is sufficient, usually 1 hr sufficing.

After the second binding step, the insoluble support is again washed free of non-specifically bound material, leaving the specific complex formed between the target protein and the specific binding member. The signal produced by the bound conjugate is detected by conventional means. Where an enzyme conjugate is used, an appropriate enzyme substrate is provided so a detectable product is formed.

Other immunoassays are known in the art and may find use as diagnostics. Ouchterlony plates provide a simple determination of antibody binding. Western blots may be performed on protein gels or protein spots on filters, using a detection system specific for the cancer associated kinase corresponding to SEQ ID NOS:1, 3, 5, 7, 9, 11 or 13 as desired, conveniently using a labeling method as described for the sandwich assay.

In some cases, a competitive assay will be used. In addition to the patient sample, a competitor to the targeted protein is added to the reaction mix. The competitor and the cancer associated kinase corresponding to SEQ ID NOS:1, 3, 5, 7, 9, 11 or 13 compete for binding to the specific blinding partner. Usually, the competitor molecule will be labeled and detected as previously described, where the amount of competitor binding will be proportional to the amount of target protein present. The concentration of competitor molecule will be from about 10 times the maximum anticipated protein concentration to about equal concentration in order to make the most sensitive and linear range of detection.

In some embodiments, the methods are adapted for use in vivo, e.g., to locate or identify sites where cancer cells are present. In these embodiments, a detectably-labeled moiety, e.g., an antibody, which is specific for the protein encoded by one of SEQ ID NOS:1, 3, 5, 7, 9, 11 or 13 is administered to an individual (e.g., by injection), and labeled cells are located using standard imaging techniques, including, but not limited to, magnetic resonance imaging, computed tomography scanning, and the like. In this manner, cancer cells are differentially labeled.

The detection methods can be provided as part of a kit Thus, the invention further provides kits for detecting the presence of an mRNA corresponding to SEQ ID NOS:1, 3, 5, 7, 9, 11 or 13, and/or a polypeptide encoded thereby, in a biological sample. Procedures using these kits can be performed by clinical laboratories, experimental laboratories, medical practitioners, or private individuals. The kits of the invention for detecting a polypeptide comprise a moiety that specifically binds the polypeptide, which may be a specific antibody. The kits of the invention for detecting a nucleic acid comprise a moiety that specifically hybridizes to such a nucleic acid. The kit may optionally provide additional components that are useful in the procedure, including, but not limited to, buffers, developing reagents, labels, reacting surfaces, means for detection, control samples, standards, instructions, and interpretive information.

### Samples for Analysis

Sample of interest include tumor tissue, e.g. excisions, biopsies, blood samples where the tumoris metastatic, etc. Of particular interest are solid tumors, e.g. carcinomas, and include, without limitation, tumors of the liver and colon. Liver cancers of interest include hepatocellular carcinoma (primary liver cancer). Also called hepatoma, this is the most common form of primary liver cancer. Chronic infection with hepatitis B and C increases the risk of developing this type of cancer. Other causes include cancer-causing substances, alcoholism, and chronic liver cirrhosis. Other liver cancers of interest for analysis by the subject methods include hepatocellular adenoma, which are benign tumors occuring most often in women of childbearing age; hemangioma, which are a type of benign tumor comprising a mass of abnormal blood vessels, cholangiocarcinoma, which originat s in the lining of the bile channels in the liver or in the bile ducts; hepatoblastoma, which is common in infants and children; angiosarcoma, which is a rare cancer that originates in the blood vessels of th liver; and bile duct carcinoma and liver cysts. Cancers originating in the lung, breast, colon, pancreas and stomach and blood cells commonly are found in the liver after they become metastatic.

Also of interest are colon cancers. Types of polyps of the colon and rectum include polyps, which are any mass of tissue that arises from the bowel wall and protrudes into the lumen. Polyps may be sessile or pedunculated and vary considerably in size. Such lesions are classified histologically as tubular adenomas, tubulovillous adenomas (villoglandular polyps), villous (papillary) adenomas (with or without adenocarcinoma), hyperplastic polyps, hamartomas, juvenile polyps, polypoid carcinomas, pseudopolyps, lipomas, leiomyomas, or other rarer tumors.

### Screening Methods

Target Screening. Reagents specific for SEQ ID NOS:1, 3, 5, 7, 9, 11 or 13 are used to identify targets of the encoded protein in tumor cells. For example, one of the nucleic acid coding sequences may be introduced into a tumor cell using an inducible expression system. Suitable positive and negative controls are included. Transient transfection assays, e.g. using adenovirus vectors, may be performed. The cell system allows a comparison of the pattern of gene expression in transformed cells with or without expression of the kinase. Alternatively, phosphorylation patterns after induction of expression are examined. Gene expression of putative target genes may be monitored by Northern blot or by probing microarrays of candidate genes with the test sample and a negative control where gene expression of the kinase is not induced. Patterns of phosphorylation may be monitored by incubation of the cells or lysate with labeled phosphate, followed by 1 or 2 dimensional protein gel analysis, and identification of the targets by MALDI, micro-sequencing, western blot analysis, etc., as known in the art.

Some of the potential target genes of the subject cancer associated kinase corresponding to SEQ ID NOS:1, 3, 5, 7, 9, 11 or 13 identified by this method will be secondary or tertiary in a complex cascade of gene expression or signaling. To identify primary targets of the subject kinase activation, expression or phosphorylation will be examined early after induction of expression (within 1-2 hours) or after blocking later steps in the cascade with cycloheximide.

Target genes or proteins identified by this method may be analyzed for expression in primary patient samples as well. The data for the subject cancer associated kinase corresponding to SEQ ID NOS:1, 3, 5, 7, 9, 11 or 13 and target gene expression may be analyzed using statistical analysis to establish a correlation.

Compound Screening. The availability of a number of components in signaling pathways allows in vitro reconstruction of the pathway, and/or assessent of kinase action on targets. Two or more of the components may be combined in vitro, and the behavior assessed in terms of activation of transcription of specific target sequences; modification of protein components, e.g. proteolytic processing, phosphorylation, methylation, etc.; ability of different protein components to bind to each other etc. The components may be modified by sequence deletion, substitution, etc. to determine the functional role of specific domains.

Compound screening may be performed using an in vitro model, a genetically altered cell or animal, or purified protein corresponding to any one of SEQ ID NOS:1, 3, 5, 7, 9, 11 or 13. One can identify ligands or substrates that bind to, modulate or mimic the action of the encoded polypeptide. Areas of investigation include the development of treatments for hyper-proliferative disorders, e.g. cancer, restenosis, osteoarthritis, metastasis, etc.

The polypeptides include those encoded by SEQ ID NOS:1, 3, 5, 7, 9, 11 or 13, as well as nucleic acids that, by virtue of the degeneracy of the genetic , are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to after a glycosylation site, a phosphorylation site or an acetylation site, or to minimize misfolding by substitution or deletion of one or more cysteine residues that are not necessary for function. Variants can be designed so as to retain or have enhanced biological activity of a particular region of the protein (e.g., a functional domain and/or, where the polypeptide is a member of a protein family, a region associated with a consensus sequence). Variants also include fragments of the polypeptides disclosed herein, particularly biologically active fragments and/or fragments corresponding to functional domains. Fragments of interest will typically be at least about 10 aa to at least about 15 aa in length, usually at least about 50 aa in length, and can be as long as 300 aa in length or longer, but will usually not exceed about 500 aa in length, where the fragment will have a contiguous stretch of amino acids that is identical to a polypeptide encoded by SEQ ID NOS:1, 3, 5, 7, 9, 11 or 13, or a homolog thereof.

Transgenic animals or cells derived therefrom are also used in compound screening. Transgenic animals may be made through homologous recombination, where th normal locus corresponding to SEQ ID NOS:1, 3, 5, 7, 9,1 11 or 13 is altered. Alternatively, a nucleic acid construct is randomly integrated into the genome. Vectors for stable integration include plasmids, retroviruses and other animal viruses, YACs, and the like. A series of small deletions and/or substitutions may be made in the coding sequence to determine the role of different exons in kinase activity, oncogenesis, signal transduction, etc. Of interest is the use of SEQ ID NOS:1, 3, 5, 7, 9, 11 or 13 to construct transgenic animal models for cancer, where expression of the corresponding kinase is specifically reduced or absent. Specific constructs of interest include antisense sequences that block expression of the targeted gene and expression of dominant negative mutations. A detectable marker, such as lac Z may be introduced into the locus of interest, where up-regulation of expression will result in an easily detected change in phenotype. One may also provide for expression of the target gene or variants thereof in cells or tissues where it is not normally expressed or at abnormal times of development By providing expression of the target protein in cells in which it is not normally produced, one can induce changes in cell behavior, e.g., in the control of cell growth and tumorigenesis.

Compound screening identifies agents that modulate function of the cancer associated kinase corresponding to SEQ ID NOS:1, 3, 5, 7, 9, 11 or 13. Agents that mimic its function are predicted to activate the process of cell division and growth. Conversely, agents that inhibit function may inhibit transformation. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains, e.g. the kinase catalytic domain, the regulatory domain, the auto-inhibitory domain, etc.

The term “agent” as used herein describes any molecule, e.g. protein or pharmaceutical, with the capability of altering or mimicking the physiological function of a cancer associated kinase corresponding to SEQ ID NOS:1, 3, 5, 7, 9, 11 or 13. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, tc. may be used. The mixture of components are added in any order that provides for the requisite binding. Incubations are performed at any suitable temperature, typically between 4 and 40° C. incubation periods are selected for optimum, activity, but may also be optimized to facilitate rapid high-throughput screening. Typically between 0.1 and 1 hours will be sufficient.

Other assays of interest detect agents that mimic the function of a cancer associated kinase corresponding to SEQ ID NOS:1, 3, 5, 7, 9, 11 or 13. For example, an expression construct comprising the gene may be introduced into a cell line under conditions that allow expression. The level of kinase activity is determined by a functional assay, for example detection of protein phosphorylation. Alternatively, candidate agents are added to a cell that lacks the functional cancer associated kinase corresponding to SEQ ID NOS:1, 3, 5, 7, 9, 11 or 13, and screened for the ability to reproduce the activity in a functional assay.

The compounds having the desired pharmacological activity may be administered in a physiologically acceptable carrier to a host for treatment of cancer, etc. The compounds may also be used to enhance function in wound healing, cell growth, etc. The inhibitory agents may be administered in a variety of ways, orally, topically, parenterally e.g. subcutaneously, intraperitoneally, by viral infection, intravascularly, etc. Topical treatments are of particular interest. Depending upon the manner of introduction, the compounds may be formulated in a variety of ways. The concentration of therapeutically active compound in the formulation may vary from about 0.1-10 wt %.

Formulations. The compounds of this invention can be incorporated into a variety of formulations for therapeutic administration. Particularly, agents that modulate activity of a cancer associated kinase corresponding to SEQ ID NOS:1, 3, 5, 7, 9, 11 or 13, or polypeptides and analogs thereof are formulated for administration to patients for the treatment of cells where the target activity is undesirably high or low, e.g. to reduce the level of activity in cancer cells. More particularly, the compounds of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intra-tracheal, etc., administration. The agent may be systemic after administration or may be localized by the use of an implant that acts to retain the active dose at the site of implantation.

In pharmaceutical dosage forms, the compounds may be administered in the form of their pharmaceutically acceptable salts, or they may also be used alone or in appropriate association, as well as in combination with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato. starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the site of disease, so that the local concentration of active agent is increased relative to the rest of the body.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 μg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

The use of liposomes as a delivery vehicle is one method of interest. The liposomes fuse with the cells of the target site and deliver the contents of the lumen intracellulary. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic lipids, such as phosphatidylcholine. The remaining lipid will normally be neutral lipids, such as cholesterol, phosphatidyl serine, phosphatidyl glycerol, and the like.

### Modulation of Enzyme Activity

Agents that block activity of cancer associated kinase corresponding to SEQ ID NOS:1, 3, 5, 7, 9, 11 or 13 provide a point of intervention in an important signaling pathway. Numerous agents are useful in reducing this activity, including agents that directly modulate expression as described above, e.g. expression vectors, antisense specific for the targeted kinase; and agents that act on the protein, e.g. specific antibodies and analogs thereof, small organic molecules that block catalytic activity, etc.

The genes, gene fragments, or the encoded protein or protein fragments are useful in therapy to treat disorders associated with defects in sequence or expression. From a therapeutic point of view, inhibiting activity has a therapeutic effect on a number, of proliferative disorders, including inflammation, restenosis, and cancer. Inhibition is achieved in a number of ways. Antisense sequences may be administered to inhibit expression. Pseudo-substrate inhibitors, for example, a peptide that mimics a substrate for the kinase may be used to inhibit activity. Other inhibitors are identified by screening for biological activity in a functional assay, e.g. in vitro or in vivo, kinase activity.

Expression vectors may be used to introduce the target gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The gene or protein may be introduced into tissues or host cells by any number of routes, including viral infection, microinjection, or fusion of vesicles. Jet injection may also be used for intramuscular administration, as described by Furth et al. (1992) Anal Biochem 205:365-368. The DNA may be coated onto gold microparticles, and delivered intradermally by a particle bombardment device, or “gene gun” as described in the literature (see, for example, Tang et al. (1992) Nature 356:152-154), where gold micro projectiles are coated with the protein or DNA, then bombarded into skin cells.

Antisense molecules can be used to down-regulate expression in cells. The antisense reagent may be antisense oligonucleotides (ODN), particularly synthetic ODN having chemical modifications from native nucleic acids, or nucleic acid constructs that express such antisense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996) Nature Biotechnology 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in vitro or in an animal model. A combination of sequences may also be used, where several regions of the mRNA sequence are selected for antisense complementation.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature, which alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural .beta.-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has, been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

MAP3K11

The Genbank database was searched for ESTs showing similarity to known kinase domain-related proteins using the “basic local alignment search tool” program, TBLASTN, with default settings. Human ESTs identified as having similarity to these known kinase domain (defined as p<0.0001) were used in a BLASTN and BLASTX screen of the GenBank non-redundant (NR) database.

ESTs that had top human hits with >95% identity over 100 amino acids were discarded. This was based upon the inventors' experience that these sequences were usually identical to the starting probe sequences, with the differences due to sequence error. The remaining BLASTN and BLASTX outputs for each EST were examined manually, i.e., ESTs were removed from the analysis if the inventors determined that the variation from the known kinase domain-related probe sequence was a result of poor database sequence. Poor database sequence was usually identified as a number of ‘N’ nucleotides in the database sequence for a BLASTN search and as a base deletion or insertion in the database sequence, resulting in a peptide frameshift, for a BLASTX output. ESTs for which the highest scoring match was to non-kinase domain-related sequences were also discarded at this stage.

Using widely known algorithms, e.g. “Smith/Waterman”, “Fasta”, “FastP”, “Needleman/Wunsch”, “Blast”, “PSIBlast,” homology of the subject nucleic add to other known nucleic acids was determined. A “Local FastP Search” algorithm was performed in order to determine the homology of the subject nucleic acid invention to known sequences. Then, a ktup value, typically ranging from 1 to 3 and a segment length value, typically ranging from 20 to 200, were selected as parameters. Next, an array of position for the probe sequence was constructed in which the cells of the array contain a list of positions of that substring of length ktup. For each subsequence in the position array, the target sequence was matched and augmented the score array cell corresponding to the diagonal defined by the target position and the probe subsequence position. A list was then generated and sorted by score and report. The criterion for perfect matches and for mismatches was based on the statistics properties of that algorithm and that database, typically the values were: 98% or more match over 200 nucleotides would constitute a match; and any mismatch in 20 nucleotides would constitute a mismatch.

Analysis of the BLASTN and BLASTX outputs identified a EST sequence from IMAGE clone AI803752 that had potential for being associated with a sequence encoding a kinase domain-related protein, e.g., the sequence had homology, but not identity, to known kinase domain-related proteins.

After identification of MAP3K11 ESTs were discovered, the clones were added to Kinetek's clone bank for analysis of gene expression in tumor samples. Gene expression work involved construction of unigene clusters, which are represented by entries in the “pks” database. A list of accession numbers for members of the clusters were assigned. Subtraction of the clusters already present in the clone bank from the clusters recently added left a list of clusters that had not been previously represented in Kinetek's clone bank. For each of the clusters, a random selection of an EST IMAGE accession numbers were chosen to keep the clusters. For each of the clusters which did not have an EST IMAGE clone, generation of a report so that clone ordering or construction could be implemented was performed on a case by case basis. A list of accession numbers which were not in clusters was constructed and a report was generateds.

The AI803752 IMAGE clone was sequenced using standard ABI dye-primer and dye-terminator chemistry on a 377 automatic DNA sequencer. Sequencing revealed that the sequence corresponds to SEQ ID NO:1.

Rapid Amplification of cDNA Ends (RACE).

The gene specific oligodeoxynucleotide primers SEQ ID NO:15 and 16 were designed and then used to construct full length MAP3K11 cDNA by 5 prime RACE (rapid amplification of cDNA ends; Frohman et al. (1988), Proc. Natl. Acad. Sci. USA 85:8898-9002).

A nested primer strategy was used on human brain cDNA provided with a Marathon-Ready™ RACE kit (Clontech, Palo Alto, Calif.). Following this, thermal cycling on a PE DNA Thermal Cycler 480 was done. When cycling was completed, the PCR product was analyzed, along with appropriate DNA size markers, on a 1.0% agarose/EtBr gel.

The product so obtained comprised a MAP3K11 polynucleotide having the sequence of SEQ ID NO:1.

Expression Analysis of MAP3K11

The expression of MAP3K11 was determined by dot blot analysis, and the protein was found to be upregulated in several tumor samples.

Dot blot preparation. Total RNA was purified from clinical cancer and control samples taken from the same patient. Samples were used from both liver and colon cancer samples. Using reverse transcriptase, cDNAs were synthesized from these RNAs. Radiolabeled cDNA was synthesized using Strip-EZ™ kit (Ambion, Austin, Tex.) according to the manufacturer's instructions. These labeled, amplified cDNAs were then used as a probe, to hybridize to human protein kinase arrays comprising human MAP3K11. The amount of radiolabeled probe hybridized to each arrayed EST clone was detected using phosphorimaging.

The expression of MAP3K11 was substantially upregulated in the tumor tissues that were tested. The data is shown in Table 1, expressed as the fold increase over the control non-tumor sample.

TABLE 1

| | liver | liver | liver | colon | colon | colon | colon | colon | colon | colon |
|---|---|---|---|---|---|---|---|---|---|---|
| Target | 1 | 2 | 3 | 1 | 4 | 5 | 7 | 8 | 9 | 10 |
| MAP3K11 | 4.1 | 1.3 | 2.3 | 2.1 | 1.1 | 1.9 | 3.4 | 1.3 | 0.9 | 1.75 |
| beta-actin | 2.05 | 1.07 | 1.57 | 0.42 | 1.28 | 2.19 | 1.20 | 4.60 | 0.60 | 0.49 |
| GAPDH | 1.30 | 0.33 | 1.25 | 0.76 | | | | | | |
| K413 | | | | | 1.72 | 2.36 | 2.10 | 1.00 | 1.00 | 1.68 |
| (ribosomal | | | | | | | | | | |
| protein) | | | | | | | | | | |

The data displayed in Table 2 provides a brief summary of the pathology report of the patient samples.

TABLE 2

| | | | | | | | Lym- | |
|---|---|---|---|---|---|---|---|---|
| | | | | Site of | | Vascu- | phatic | |
| | | | Precursor | Involve- | | lar Inva- | Involve- | Meta- |
| Patient | Age | Gender | Adenoma | ment | Differentiation | sion | ment | stasis |
| Liver | 49 | Female | N/a | Liver | Moderately | No | Yes | No |
| 1 | | | | | Differentiated | | | |
| Liver | 53 | Male | N/a | Liver | Moderately | Yes | No | No |
| 2 | | | | | Differentiated | | | |
| Liver | 75 | Female | Adenoma | Right | Moderately | No | No | No |
| 3 | | | | Colon | differentiated | | | |
| Colon | 55 | Female | No | Rectum | Moderately | N/A | Yes | No |
| 1 | | | | | Differentiated | | | |
| Colon | 91 | Female | Adenoma | Cecum | Moderately | No | Yes | No |
| 4 | | | | | Differentiated | | | |
| Colon | 79 | Male | No | Ileum | | | | |
| 5 | | | | and | | | | |
| | | | | Colon | | | | |
| Colon | | | | | Moderately | No | No | No |
| 7 | | | | | Differentiated | | | |
| Colon | 61 | Male | Yes | | Moderately | No | Yes | Yes, |
| 8 | | | | | Differentiated | | | Liver |
| Colon | 60 | Male | No | Recto- | Moderately | Yes | No | Yes, |
| 9 | | | | Sigmoid | Differentiated | | | Liver |
| Colon | 60 | Male | No | Sigmoid | Moderately | Yes | Yes | No |
| 10 | | | | Colon | Differentiated | | | |

### Example 2

CaMK-X1

The Genbank database was searched for ESTs showing similarity to known kinase domain-related proteins using the “basic local alignment search tool” program, TBLASTN, with default settings. Human ESTs identified as having similarity to these known kinase domain (defined as p<0.0001) were used in a BLASTN and BLASTX screen of the GenBank non-redundant (NR) database, searched against the sequence of the catalytic domain of CaMK-I (Genbank hs272I161). Sequence screening was performed as described in Example 1.

Analysis of the BLASTN and BLASTX outputs identified an EST sequence from IMAGE clone AA838372 that had potential for being associated with a sequence encoding a kinase domain-related protein, e.g., the sequence had homology, but not identity, to known kinase domain-related proteins. Further, CaMK-X1 was found to have sequence similarity to members of the calmodulin dependent protein kinase family. The reported nucleotide sequence of the 5′ EST of the AA838372 IMAGE clone corresponds approximately to 400 nucleotides of SEQ ID NO:1. A search of the UniGene database revealed that the 5′ EST of the AA838372 IMAGE done represented a novel human gene.

The AA838372 IMAGE clone was sequenced using standard ABI dye-primer and dye-terminator chemistry on a 377 automatic DNA sequencer. Sequencing revealed that the sequence corresponds to nucleotides 1 to 2447 of SEQ ID NO:3. Analysis of this gene fragment revealed that the gene product is a novel kinase domain-related protein, thereafter termed CaMK-X1.

Rapid Amplification of cDNA Ends (RACE).

The gene specific oligodeoxynucleotide primer 5′-GGAGGGCG AGGAAACTGGGGAAG-3′ (SEQ ID NO:17) was designed and then used to construct full length CaMK-X1 cDNA by 5 prime RACE (rapid amplification of cDNA ends; Frohman et al. 1988, Proc. Natl. Acad. Sci. USA 85:8898-9002). Adaptor primer (AP1) was used as sense primer, and SEQ ID NO:3 was used as antisense primer. A nested primer strategy was used on fetal brain cDNA provided with a Marathon-Ready™ RACE kit (Clontech, Palo Alto, Calif.). Following this, thermal cycling on a PE DNA Thermal Cycler 480 was done. When cycling was completed, the PCR product was analyzed, along with appropriate DNA size markers, on a 1.0% agarose/EtBr gel.

The product so obtained comprised a CaMK-X1 polynucleotide having the sequence of SEQ ID NO:3. BLASTX analysis indicated that the starting methionine residue was present at nucleotide 10, and that an upstream in-frame stop codon was present at nucleotide 1498, and the longest ORF (SEQ ID NO:3) predicted a protein of 476 amino acids (SEQ ID NO:4).

Homology analysis of the deduced amino acid sequence of CaMK-X1 revealed strong sequence identity with CaMK I from amino acid residues 11 to 333. The corresponding region of CaMK I contains the threonine residue required for activation and the regulatory domain that folds over the active site unless bound by CaM (Matsuchita et al. (1998) Journal of Biological Chemistry 273, 21473-21481). CaMK-X1 also has a region between residues 23 and 277 that is highly homologous (46% identity) to the highly conserved serine/threonine kinase active site.

Expression Analysis

The expression of CaMK-X1 was determined by Northern Blot, and dot blot analysis, and the protein was found to be upregulated in several tumor samples. In normal tissue, CaMK-X1 is highly expressed in brain, and at lower levels in kidney and spleen.

Dot blot preparation. Total RNA was purified from clinical cancer and control samples taken from the same patient. Samples were used from both liver and colon cancer samples. Using reverse transcriptase, cDNAs were synthesized from these RNAs. Radiolabeled cDNA was synthesized using Strip-EZ™ kit (Ambion, Austin, Tex.) according to the manufacturer's instructions. These labeled, amplified cDNAs were then used as a probe, to hybridize to human protein kinase arrays comprising human CaMK-X1. The amount of radiolabeled probe hybridized to each arrayed EST clone was detected using phosphorimaging.

The expression of CaMK-X1 was substantially upregulated in the tumor tissues that were tested. The data is shown in Table 3, expressed at the fold increase over the control non-tumor sample.

TABLE 3

| | liver | liver | liver | colon | colon | colon | colon | colon | colon | colon |
|---|---|---|---|---|---|---|---|---|---|---|
| | 1 | 2 | 3 | 1 | 2 | 3 | 4 | 5 | 6 | 7 |
| CaMK-X1 | 5.0 | 4.9 | 5.1 | 2.3 | 2.6 | 1.5 | 3.3 | 1.2 | 1.3 | 4.05 |

Functional Assays

A deletion mutant clone was created to aid in the characterization of this kinase in vivo. In addition, it is shown that CaMK-X1 phosphorylates CREB at Ser 133 in Jurkat cells, and this phosphorylation is controlled by a Calmodulin binding site.

CaMK-X1 kinase activity was shown in vitro using three different approaches. CaMK-X1 was purified from Hi5 insect cells and HEK293 cells overexpressing CaMK-X1 using GST and Ni2+ affinity chromatography. Furthermore, CaMK-X1 was purified via immunoprecipitation using a monoclonal antibody directed against the X-press fusion protein. CaMK-X1 displays no activity toward exogenous substrates in the absence of Ca2+ and calmodulin. In the presence of Ca2+ and calmodulin, CaMK-X1 phosphorylated Syntide and CREBtide peptides. This is the first experimental demonstration that CaMK-X1 behaves as a calcium/calmodulin-dependent protein kinase.

Cloning and sub-cloning. Cloning of CaMK-X1 and construction of cDNA expression vectors and the CaMK-X1 deletion mutant A human brain cDNA library was used with a 5′ RACE system. To generate the full-length cDNA of CaMK-X1, a pair of primers were designed and used in the PCR reaction. (SEQ ID NO:24) 5′-GTGGAGGGC GAGGAAACTGGGGAAG-3 and (SEQ ID NO:25) 5′-CTCGAGTCACA TAATGAGACAGACTCCAGTC. The coding area of CaMK-X1 was amplified using the above pair of primers. The amplification product was then cloned into a Promega T/A vector and subsequently cloned into other vectors as necessary. The EcoRI and XhoI fragment of CaMK-X1 was cloned into bacterial expression vector pGEX4T-3 and mammalian expression vector pcDNA3.1/His B. All constructs were verified by restriction enzyme digestion and DNA sequencing.

Tissue distribution of CaMK-X1. CaMK-X1 was used to probe and blot mRNA, using a commercially available poly-A+ selected blot (Clontech, Palo Alto, Calif.), and hybridized according to the manufacturer's instructions. The CaMK-X1 clone (corresponding to SEQ ID NO:3) was radiolabeled using Strip-EZ PCR kit (Ambion, Austin, Tex.) according to the manufacturer's instructions.

It was found that in normal tissues, CaMK-X1 is expressed at high levels only in the brain, hybridizing to an mRNA of approximately 2.8 Kb in length. The mRNA was expressed at low levels in the kidney and spleen. The mRNA in the Northern blot ran at a position consistent with a molecular weight between 2.5-2.7 kb.

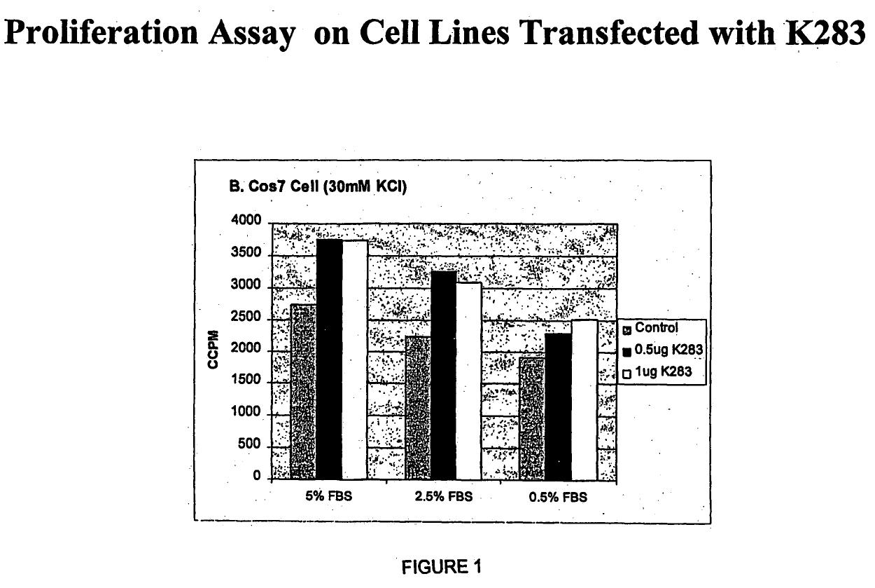

CaMK-X1 increases proliferation of Cos7 cells. The proliferation rate of Cos7 cells when transfected with CaMK-X1 was examined. To determine whether increased levels of CaMK-X1 had any effect on cell proliferation, Cos7 cells were transfected with increasing concentrations of CaMK-X1 or vector plasmids in the presence of KCl. Cell proliferation was measured by standard protocols. As shown in FIG. 1, transfection of CaMK-X1 increased the rate of proliferation, whereas the same concentration of vector alone decreased the rate of proliferation. The proliferation rate of Cos7 cells transiently transfected with CaMK-X1 is higher in 5% serum that at the 2.5% or 0.5%, suggesting that CaMK-X1 induced proliferation is modulated by serum. This data demonstrates that CaMK-X1 can promote cell proliferation.

CaMK-X1 phosphorylates CREB in vivo. cAMP response element-binding protein (CREB) is a DNA binding transcription factor. A number of growth factors and hormones have been shown to stimulate the expression of cellular genes by inducing the phosphorylation of the nuclear factor CREB at Ser 133 (Montminy (1997) Annu.Rev. Biochem. 66:807-822). Originally characterized as a target for PKA-mediated phosphorylation, CREB is also recognized by other kinases including Protein kinase C, calmodulin kinase, microtubule-activated protein kinase activated protein, and protein kinase B/AKT.

It was investigated whether CaMK-X1 could regulate CREB-Ser 133 phophorylation in vivo. To analyze CaMK-X1 in vivo, Jurkat cells were utilised. Jurkat cells transfected with various concentrations of plasmids carrying CaMK-X1 or vector were stimulated with KCl. Whole cell protein was prepared from these transfected cells and the phosphorylation status of CREB at Ser 133 was determined. Detection of CREB phosphorylation was carried out using anti-phospho-CREB antibody. Phosphorylation of CREB increased with increasing amounts of the CaMK-X1 gene transfection, but only in the presence of Ca²⁺.

To assess the effects of intracellular Ca²⁺ on CaMK-X1, transfected Jurkat cells were treated with 30 mM KCl. KCl depolarizes cell membranes thereby creating an increase in Intracellular Ca²⁺. Addition of KCl resulted in significant phosphorylation of CREB only in cells transfected with CaMK-X1. These results show that CaMK-X1 is activated by Ca²⁺ e and subsequently phosphorylates CREB at Ser 133 in Jurkat cells.

Calmodulin binding site deletion mutant of CaMK-X1 constitutively phosphorylates CREB in vivo. It has been shown previously that CaM kinases can be made Ca²⁺ independent by truncation of the calmodulin binding site. Similarly, a constitutively active form of CaMK-X1was created by removing the putative CaM-binding domain via truncation at amino acid Gin 301. This deletion site eliminates the two predicted Ca²⁺/Calmodulin-binding sites in the autoinhibitory domain. The truncated gene was placed in a pcDNA mammalian expression vector for transfection experiments.

To analyze the function of the mutant CaMK-X1 in vivo, Jurkat cells were used. Jurkat cells transfected with various concentrations of plasmids carrying CaMK-X1 or vector were stimulated with KCl. Whole cell protein was prepared from these transfected cells and the phosphorylation status of CREB at Ser 133 was determined. Detection of CREB phosphorylation was carried out using anti-phospho-CREB antibody. Mock treatment by the vectors did not have any effect on CREB phosphorylation. The transfection of wild type CaMK-X1 had no effect on CREB phosphorylation; however, addition of KCl to wild type transfected Jurkat cells resulted in significant CREB phosphorylation. Transfection of the deletion mutant had a significant effect on CREB phosphorylation without the addition of KCl. These results demonstrate that truncation of wild type CaMK-X1 at Gin 301 converted the enzyme to a Ca²⁺/CaM-independent state.

Expression of CaMK-X1 kinase in HEK293 cells. The availability of the CaMK-X1 clone allows us to reconstruct the signaling pathway. This allows us to identify downstream compon nts such as transcription factors or modification of protein components such as phosphorylation, proteolytic processing, methylation, and the like, which finds use in drug screening.

To characterize CaMK-X1 at the protein level, HEK293 cells were transfected with pcDNA3-Xpress (Invitrogen) containing the CaMK-X1 coding sequence fused to the Xpress epitope; and stable cell lines were created using standard techniques. Five stable cell lines containing th pcDNA-CaMK-X1 plasmid and five containing the vector only control were selected and CaMK-X1 expression levels were determined. Whole cell extracts were prepared from each cell line. The cell lysates were analysed by Western blotting with an anti Xpress monoclonal antibody. These experiments revealed a 53 kDa fusion protein present in the CaMK-X1 transfected cells that was absent in the control cells.

The transfected HEK293 cells stably expressed CaMK-X1 as an Xpress fusion protein. Similarly, we have detected a GST-CaMK-X1 fusion protein expressed in Hi5 cells. Glutathione-sepharose affinity chromatography was used to purify the GST-CaMK-X1 fusion protein. Glutathione-sepharose purified CaMK-X1 and anti-Xpress antibody immunoprecipitated CaMK-X1 were subjected to Western blot analysis. This Western blot indicates that CaMK-X1 can be purified from both transfected HEK293 cell lysate and Hi5 cell lysate. These methodologies were used to purify CaMK-X1 for further characterization.

A protein with a molecular mass of 53 kDa was identified when lysates of HEK293 cells transfected with the Xpress-CaMK-X1 clone were subjected to immunoprecipitation with anti-Xpress antibody followed by anti-X-press Western blotting, which band was absent with vector alone transfected cells. This data confirms that the anti-X-press antibody selectively immunoprecipitated the fusion protein (X-press-CaMK-X1).

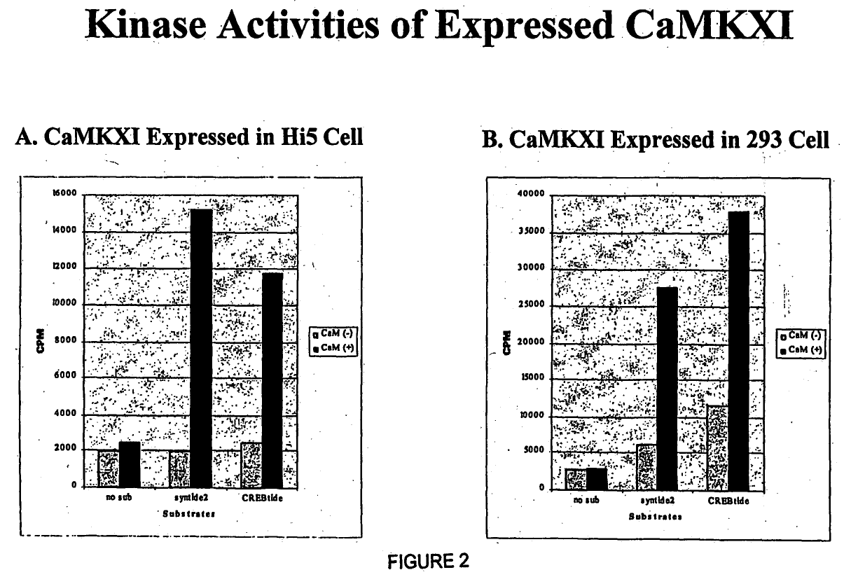

These immunoprecipitated materials were assayed for kinase activity, using the peptides (SEQ ID NO:26) CREBtide: Lys Arg Arg Glu lie Leu Ser Arg Arg Pro Ser Tyr Arg; (SEQ ID NO:27) Syntide 2: Pro Leu Ala Arg Thr Leu Ser Val Ala Gly Leu Pm Gly Lys Lys; and (SEQ ID. NO:28) Calmodulin Dependent Protein Kinase Substrate: Pro Leu Ser Arg Thr Leu Ser Val Ser Ser. The immunoprecipitated materials were subjected to an in vitro kinase assay as described above. Since it was shown that CaMK-X1 phosphorylates CREB in vivo, it was reasoned that CaMK-X1 would phosphorylate CREBtide and Syntide 2 (Colbran et al. (1989) J Biol Chem 264(9):4800-4804). As predicted, CaMK-X1 phosphorylated CREBtide and Syntide 2 in vitro. In contrast, CaMK-X1 could not phosphorylate control peptide. The degree of phosphorylation is augmented in the presence of calmodulin, as shown in FIG. 2. In the absence of a substrate, there is no significant incorporation of radioactive material (³²P) indicating that CaMK-X1 does not autophosphorylate under these assay conditions. This demonstrates that immunoprecipitated CaMK-X1 possesses a kinase activity and that this kinase activity is capable of phosphorylating peptides in vitro. These studies also revealed that CaMK-X1 requires calmodulin for efficient activity.

Catalytic activity and comparison of substrate specificities of CaMK-X1. In order to determine if CaMK-X1 is an active kinase in vitro, the clone was Histidine tagged, expressed in Sf9 cells and purified with a Ni2+ affinity column. For analysis of substrate specificity, we tested the following three peptides; CREBtide, Syntide 2 and CDPK-peptide (control peptide). In vitro kinase assays were then performed. As described above, CREBtide and Syntide 2 are phosphorylated by the purified CaMK-X1. The rate of phosphorylation is increased in the presence of Ca²⁺ and calmodulin. Compared to a no substrate control, addition of the peptides resulted in significant ³²p incorporation. These results indicate that CaMK-X1 phosphorylates these peptides in vitro. Our studies also revealed Syntide 2and CREBtide had higher incorporation of ³²p than the control peptide. These observations further confirm the in vivo data.

Summary. We have demonstrated that CaMK-X1 phosphorylates CREB in cells and in vitro at Ser 133. We have also demonstrated CaMK-X1 kinase activity in vitro. We were able to purify CaMK-X1 from transfected Hi5 insect cells and from a HEK293 cell line overexpressing CaMK-X1 using glutathione-sepharose and Ni2+ affinity chromatography. Furthermore, CaMK-X1 was purified by immunoprecipitation using a monoclonal antibody directed against the Xpress fusion protein. CaMK-X1 displays no activity toward exogenous substrates in the absence of Ca²⁺ and calmodulin. in the presence of Ca²⁺ and calmodulin, CaMK-X1 phosphorylated Syntide 2 and CREBtide. These results indicate that CaMK-1 are involved in human pathology.

Materials.

Dulbecco's Modified Eagle Medium (DMEM), RPMI Medium 1640, L-glutamine, phosphate buffered solution (PBS), fetal bovine serum (FBS), and restriction enzymes were from GibcoBRL. TOPO cloning kit (including PCR materials and pCR 2.1-Topo vector) were from Invitrogen. Phospho-CREB (Ser133) polyclonal rabbit antibody was from Cell Signaling Technology. 96- and 6-well delta surface plates were from NUNCLON. QIAprep Spin Miniprep Kit was from Qiagen. Wizard Plus Minipreps DNA Purification System (for gel extractions) (Promega). FuGENE 6 Transfection Reagent was from Boehringer Mannheim. pcDNA3.1 mammalian expression vector (Invitrogen). Western Blotting Luminol Reagent was from Santa Cruz Biotechnology. 2° goat-anti-rabbit IgG (H+L) HRP conjugated antibody was from Bio-Rad Laboratories.

Cloning of full length CaMK-X1. To generate the full-length cDNA of CaMK-X1, a pair of primers were designed and used in the PCR reaction. (SEQ ID NO:29) 5′-GAATTCAATGGGTCGAAAGGAAGAAGATGA and (SEQ ID NO:25) 51′-CTCGAGTCACATAATGAGACAGACTCCAGTC. The amplification product was cloned into cloning vectors through restriction sites EcoRI and XhoI. The EcoRI and XhoI fragment was cloned into bacteria expression vector pGEX-4T-3 and mammalian expression vector pcDNA3.1/HisB. All constructs were verified by restriction enzyme digestion and DNA sequencing.

Construction of deletion mutant CaMK-X1CA. A deletion mutant was created using these oligonucleotides EcoR1 (SEQ ID NO:30) 5′-GAATTCAATGGGTCGAAAGGMGAAGATGA-3′ forward, and Xho1 (SEQ ID NO:31) 5′-CTCGAGCTGGATCTGGAGGCTGACTGATGG-3′ rev rse. The resulting PCR fragment was cloned into mammalian expression vector pcDNA 3.1.

Cell Culture. Cells were incubated at 37° C. in 5% CO₂ (standard conditions). All cells, unless mentioned below, were cultured in DMEM with FBS; the specific amount of FBS varies and is stated in the report for each result. Jurkat cells were cultured in RPMI Medium 1640 with added glucose, L-glutamine, and 10% FBS.

Cell Transfection. Cells were seeded to a density of 2×10⁵ in 6 well plates (in appropriate media for the particular cell line) and incubated for 24 hours under standard conditions. 3 ml of FuGENE 6 transfection reagent was diluted in 97 ml of serum-free media (appropriate for the cell line being transfected) and left for 5 minutes at room temperature; that was then added drop-wise to th desired amount of plasmid DNA (in pcDNA3.1) and left for 10 minutes at room temperature. The finished transfection solution was then added drop-wise to the cells, which were then incubated for 24 hours under standard conditions.

Proliferation Assay. The media from 6 well plates was removed and trypsin was added to digest the extracellular matrix holding the cells to the plate; media (appropriate to the cell type) was then added to deactivate the trypsin. The cells and media were transferred into Falcon tubes, centrifuged, and the supematant was discarded. The cells were resuspended in appropriate media. 3000 cells were seeded in each well of a 96 well plate and appropriate media was added up to 90 ml.

Ten μl of 0.1 Ci/L ³H-thymidine was added to each well. The plates were then incubated for 24 hours under standard conditions. Twenty-five μl of cold trichloroacetic acid was added to each well and the plates were kept at 4° C. for 2 hours. The plates were then washed in cold running water and allowed to dry. Proliferation was determined by incorporation of thymidine as measured via scintillation counting.

Cell lysis. Lysis buffer was 50 mM Hepes (pH 7.5), 150 mM NaCl, 1% NP-40, 2 mM NaF, 1 mM Na₃VO₄, 1 mM PMSF, 1 mg/ml pepstatin, 1 mg/ml leupeptin, 1 mg/ml aprotinin, and 20 mM β-glycerophosphate. For adherent cells, the media was removed from the 6 well plate, the wells were washed with PBS which was then removed, the plates were put on ice and 40 ml of lysis buffer was then added to each well. Crude lysates were collected with a cell scraper and placed in an Eppendorf tube. For non-adherent cells, the media and cells were transferred from a 6-well plate to tubes, centrifuged and the supematant removed; 40 ml of lysis buffer was then added. All crude lysates were then vortexed and left on ice for 10 minutes. The crude lysates were centrifuged at 14,000 RPM for 10 minutes at 4° C. and the supematant, the final lysate, was transferred to new tubes.

Western Blotting. Equal weights of cell lysate proteins were mixed with 4× loading buffer, boiled for five minutes and were then briefly centrifuged. The samples were run on a 10% SDS-PAGE and were then transferred to PVDF membranes which were washed with TTBS and blocked with 2% BSA. They were blotted with primary antibody for 16 hours at 4° C. The membranes were washed with TTBS, blotted with secondary antibody for 1 hour and washed with TTBS. The luminol reagent was added, the blot was placed on film and the autoradiograph developed.

Expression and purification of CaMK-X1 protein. The human CaMK-XI gene (K283) was sub-cloned into baculovirus transfer vector pAcG4T3 derived from pAcG2T (BD Biosciences) under the control of the strong AcNPV (Autograpga californica Nuclear Polyhedrosis Virus) polyhedrin promoter. This was co-transfected with linear BaculoGold DNA in Spodoptera frugiperda Sf9 cells following standard procedure (BD Biosciences). T he GST-CaMK-X1 recombinant baculovirus was amplified in Sf9 cells in TNM-FH medium (JHR Biosciences) with 10% fetal bovine serum. The GST-CaMK-X1 protein was expressed in approximately 5×10⁸ a Hi-5 cells (Invitrogen) in 500 ml of Excell-400 medium (JHR Biosciences) at a multiplicity of infection (MOI) of five for a period of 72 h in a spinner flask. The cells were harvested at 800×g for 5 min at 4° C. The pellet was lysed in 40 ml of Lysis Buffer (50 mM Tris-HCl, PH7.5, 2.5 mM EDTA, 150 mM NaCl, 1% NP-40, 0.1% β-mercaptoethanol, 10 μg/ml DNase I, 0.5 mM sodium orthovanadate, 50 mM β-glycerophosphate, 0.1 mM PMSF, 1 mM benzamidine, 2 μg/ml aprotinin, 2 μg/ml leupeptin, 1 μg/ml pepstatin) by sonication and centrifuged at 10,000×g at 4° C. for 15 min. The supematant was loaded on a column containing 2.5 ml of glutathione-sepharose (Sigma). The column was washed with Wash Buffer A (50 mM Tris-HCl, pH 7.5, 1 mM EDTA, 500 mM NaCl, 0.1% β-mercaptoethanol, 0.1% NP-40, 0.1 mM sodium orthovanadate, 50 mM β-glycerophosphate, 0.1 mM PMSF, 1 mM benzamidine) until OD280 returned to baseline, then Wash Buffer B (50 mM Tris-HCl, PH7.5, 1 mM EDTA, 50 mM NaCl, 0.1% β-mercaptoethanol, 0.1 mM PMSF). The GST-CaMK-X1 protein was eluted in Elution Buffer (50 mM Tri-HCl, PH7.5, 1 mM EDTA; 50 mM NaCl, 0.1% β-mercaptoethanol, 10 mM glutathione, 10% glycerol). The fraction was aliquoted and stored at −70° C.

CaMK-XI in vitro assay. CaMK-X1 was assayed at room temperature for 15 min in 50 mM HEPES, pH 8.0, 10 mM MgCl₂, 1 mM dithiothreitol, 0.005% Tween 20, 1 mM CaCl₂, 1.5 mM calmodulin (CalBiochem), 50 uM [γ-³²P]-ATP and 0.2 μg/μl Syntide 2 (American Peptide Company) or CREBtide (CalBiochem) in a final volume of 25 μl. Reactions were initiated by addition of [γ-³²P]-ATP and terminated by spotting 10 μl of the reaction mixture onto P81 paper followed by washing in 1% phosphoric acid.

Immunoprecipitation. For immunoprecipitations, HEK293 cells in 35 mm dishes stably expressing CaMK-X1-X-press plasmid were washed twice in ice-cold PBS and lysed in solution containing 50 mM Tris/HCl, pH 7.6, 2 mM EGTA, 2 mM EDTA, 2 mM dithiothreitol, protease inhibitors aprotinin (10 μg/ml) leupeptin (100 μg/ml) pepstatin (0.7 μg/ml), 1 mM 4-(2-aminoethyl) benzenesulfony fluoride hydrochloride, and 1% Triton X-100 (Lysis buffer). Proteins were immunoprecipitated with the anti-X-press antiserum (1:100 dilution) or with control serum. The immuno complexes were recovered using protein G Sepharose.

In vitro kinase assay with immunoprecipitated materials. CaMK-X1 was eluted from the immunocomplexes as described in the previous section and 20 μl of the eluate was mixed with 20 μl of phosphorylation mix containing 100 μM [γ ³²P] ATP (specific activity, 400-600 cpm/pmol, 30 mM Tris, pH 7.4, 30 mM MgCl₂, 1 mM DTT, and 250 nM peptide and incubated for 10-15 minutes at 30° C.

Northern Blot analysis. Northern blot analysis was performed using an [α ³²P] dCTP-labeled CaMK-X1 cDNA fragment corresponding to bases 1.2 kb of human CaMK-X1 according to standard procedures (Ambion). RNA from several primary human tissues was analyzed with commercially available poly(A) + RNA blots (CLONTECH) The blotted membrane was dried and autoradiographed.

CaMK-X1 activity assay. Equivalent concentrations of purified CaMK-X1 preparations were incubated using a Beckman Biomek 2000 robotic system. Each well (96 well microtiter plate) contained 15 μl reaction mixture composed of 50 mM HEPES, pH 8.0, 10 mM MgCl₂, 1 mM dithiothreitol, 0.005% Tween 20, 1 mM CaCl₂, 1.5 mM Calmodulin (CalBiochem) 50 μM γ-³²P ATP (200 cpm/pmol) and 0.2 μg/μl Syntide 2 (American Peptide Company) or CREBtide (CalBiochem) in a final volume of 25 μl. The reaction was initiated by addition of [γ³²-P]-ATP and terminated by spotting 10 μl of the reaction mixture into a 96 well Millipore Multiscreen plate. The Multiscreen plate was washed in 1% phosphoric acid, dried and counted in a Wallac Microbeta scintillation counter.

### Example 3

SGK2α

The Genbank EST database was searched as described in Example 1. Analysis of the BLASTN and BLASTX outputs identified a EST sequence from IMAGE clone AF169034 that had potential for being associated with a sequence encoding a kinase domain-related protein, e.g., the sequence had homology, but not identity, to known kinase domain-related proteins.

The AF169034 IMAGE clone was sequenced using standard ABI dye-primer and dye-terminator chemistry on a 377 automatic DNA sequencer. Sequencing revealed that the sequence corresponds to SEQ ID NO:5, SGK2α. The expression of SGK2α was determined by dot blot analysis, and the protein was found to be upregulated in several tumor samples. SEQ ID NO:18 and 19 were used in amplification.

Dot blot preparation. Total RNA was purified from clinical cancer and control samples taken from the same patient. Samples were used from both liver and colon cancer samples. Using reverse transcriptase, cDNAs were synthesized from these RNAs. Radiolabeled cDNA was synthesized using, Strip-EZ™ kit (Ambion, Austin, Tex.) according to the manufacturers instructions. These labeled, amplified cDNAs were then used as a probe, to hybridize to human protein kinase arrays comprising human SGK2α. The amount of radiolabeled probe hybridized to each arrayed EST clone was detected using phosphorimaging.

The expression of SGK2α was substantially upregulated in the tumor tissues that were tested. The data is shown in Table 4, expressed at the fold increase over the control non-tumor sample.

TABLE 4

| | liver | liver | liver | colon | colon | colon | colon | colon | colon | colon |
|---|---|---|---|---|---|---|---|---|---|---|
| | 1 | 2 | 3 | 1 | 4 | 5 | 7 | 8 | 9 | 10 |
| SGK2α | 3.6 | 2.4 | 1.1 | 1.1 | 1.0 | 3.9 | 1.8 | 1.4 | 0.7 | 2.55 |
| beta-actin | 2.05 | 1.07 | 1.57 | 0.42 | 1.28 | 2.19 | 1.20 | 4.60 | 0.60 | 0.49 |
| GAPDH | 1.30 | 0.33 | 1.25 | 0.76 | Not | Not | Not | Not | Not | Not |
| | | | | | done | done | done | done | done | don |
| K413 | Not | Not | Not | Not | 1.72 | 2.36 | 2.10 | 1.00 | 1.00 | 1.68 |
| (ribosomal | done | done | done | done | | | | | | |
| protein) | | | | | | | | | | |

The data displayed in Table 5 provides a brief summary of the pathology report of the patient samples.

TABLE 5

| | | | | | | | Lym- | |
|---|---|---|---|---|---|---|---|---|
| | | | | Site of | | Vascu- | phatic | |
| | | | Precursor | Involve- | | lar Inva- | Involve- | Meta- |
| Patient | Age | Gender | Adenoma | ment | Differentiation | sion | ment | stasis |
| Liver | 49 | Female | N/a | Liver | Moderately | No | Yes | No |
| 1 | | | | | Differentiated | | | |
| Liver | 53 | Male | N/a | Liver | Moderately | Yes | No | No |
| 2 | | | | | Differentiated | | | |
| Liver | 75 | Female | Yes | Right | Moderately | No | No | No |
| 3 | | | | Colon | differentiated | | | |
| Colon | 55 | Female | No | Rectum | Moderately | N/A | Yes | No |
| 1 | | | | | Differentiated | | | |
| Colon | 91 | Female | Yes | Cecum | Moderately | No | Yes | No |
| 4 | | | | | Differentiated | | | |
| Colon | 79 | Male | No | Ileum | Moderately | No | No | No |
| 5 | | | | and | Differentiated | | | |
| | | | | Colon | | | | |
| Colon | 93 | Male | No | Recto- | Moderately | No | No | No |
| 7 | | | | Sigmoid | Differentiated | | | |
| Colon | 61 | Male | Yes | Yes | Moderately | No | Yes | Yes, |
| 8 | | | | | Differentiated | | | Liver |
| Colon | 60 | Male | No | Recto- | Moderately | Yes | No | Yes, |
| 9 | | | | Sigmoid | Differentiated | | | Liv r |
| Colon | 60 | Male | No | Sigmoid | Moderately | Yes | Yes | N |
| 10 | | | | Colon | Differentiated | | | |

Creation of stable cell lines over expressing SGK2 in HEK293 cells. We constructed a mammalian expression vector encoding N-terminal X-press tagged forms of the 45 kDa SGK2 kinase. The ORF of SGK2 was placed in frame with N-terminal Xpress and a Histidine tag in pcDNA 3 mammalian expression vector using standard PCR-based cloning techniques. To characterize SGK2 at the protein level, HEK293 cells were transfected and a stable cell line selected with pcDNA 3 His-X-press-SGK2 plasmid in the presence of G418. HEK293 cells were stably transfected with mammalian vector incorporating SGK2 to produce clones over expressing wild type SGK2.

Briefly, cells were grown in d-MEM containing 5% FCS, 2 mm L-glutamine, glucose (3.6 mg/ml) and G418 (40 μg/ml) was added to transfected cells to maintain selection pressure. The cell lysates were prepared from stable cell lines and subjected to Western blotting with anti-Xpress mAb and anti-His-antibody. A protein with a 45 kDa molecular mass was identified in lysates of HEK293 cells stably expressing SGK2. A similar protein could not be detected in the control. HEK293 cells. This analysis suggests that HEK293 cells are overexpressing SGK2 as a fusion protein. To determine whether these cells express higher levels of SGK2 mRNA, we isolated mRNA from stable cell lines as well as control HEK293 cells. Equal amounts of mRNA were immobilized on a nylon membrane and subjected to hybridization with a specific SGK2 probe. Stable cell lines expressed a significantly higher concentration of SGK2 mRNA as compared to control HEK293 cells. These results indicate that stable cell lines are over expressing SGK2 mRNA as well as SGK2 protein. These stable cell lines were used in the subsequent experiments.

Overexpressed SGK2 can phosphorylate GSK3 in vivo. We explored the identification of the downstream effectors of SGK2 by using SGK2 ovexpressing cells. SGKs have 54% nucleotide sequence homology to PKB and it has previously been shown that PKB could phosphorylate GSK3 in vivo and in vitro. In view of this, we wanted to determine whether SGK2 could regulate the activity of GSK3, a kinase that is normally phosphorylates beta catenin. GSK3 phosphorylates beta catenin and targets it for destruction via a ubiquitin-proteasome pathway. To determine whether SGK2 could phosphorylate GSK3, initially, we carried out transient transfection assays in human embryonal kidney epithelial cells (HEK293). Transfection of SGK2 resulted in increased phosphorylation of GSK3. This was monitored by specific anti-GSK3 phospho Ser9 antibody. These results suggest that SGK2 effects the phosphorylation of GSK3 in vivo.

As a control, we measured the concentration of GSK3 protein in the assay. The concentration of GSK3 is not affected by SGK2 but the phosphorylation status of GSK3 is affected by the expression of SGK2. This is particularly significant at the lower concentration of serum (0.5%) and 0.1-02 μg concentration of SGK2 plasmid. Because GSK3 activity can be inhibited by phosphorylation, it is possible that inhibition of GSK3 by SGK2 could lead to other downstream effects. To further evaluate the link between SGK2 and GSK3 we measured the phosphorylation status of GSK3 in HEK293 cells and in HEK293 cells stably transfected with SGK2 (named SGK-37A). SGK-37A cells overexpressing SGK2 had significantly higher phospho GSK3 than normal HEK293 cells.

This data demonstrates that SGK2 can modulate the phosphorylation status of GSK3 in stably transfected HEK293 cells. It has been shown that GSK3 phosphorylation leads to GSK3 inactivation (Cross et al. (1995) Nature 378:785-789). SGK2 may directly phosphorylate GSK3 and inactivate it, thereby abolishing phosphorylation of the cytoplasmic signaling molecule β-catenin and causing its stabilization and nuclear translocation. In the nucleus, β-catenin associates with TCF4 to induce the transcription of several genes including cyclin D1.

SGK2 enhances cell proliferation. Since we have shown that overexpression of SGK2 stimulates GSK3 phosphorylation, it was investigated whether this could lead to cell proliferation. To study the effects of SGK2 on cell proliferation, we used several cells types. These cells were transiently transfected with SGK2 or control DNA plasmids. The DNA synthesis rate was determined by measuring [³H] thymidine incorporation. When HEK293 and 3T3 cells were transfected with SGK2, they exhibited greater amounts of DNA synthesis than the control vector. The rate of proliferation was dependent on the concentration of transfected SGK2 plasmid. This data indicates that SGK2 stimulates cell proliferation in these cell types. Co-expression of PDK1 with SGK further enhanced the rate of proliferation.

These data reveal that SGK2 promotes proliferation in a variety of cells, and suggest that SGK2 promotes cell proliferation and support tumor progression in these types of cells.

SGK overexpression stimulates AP1 transactivation. It has previously been shown that GSK3 phosphorylates c-Jun at C-terminal sites, resulting in inhibition of DNA binding (Nikolakaki et al. (1993) Oncogene 8:833-840) This can lead to the inhibition of AP1 activity in intact cells. It is believed that this keeps the cell's homeostasis in control. Since we have shown that SGK2 phosphorylates GSK3, we wanted to evaluate whether this could modulate the AP1 transactivation in cells overexpressing SGK2.

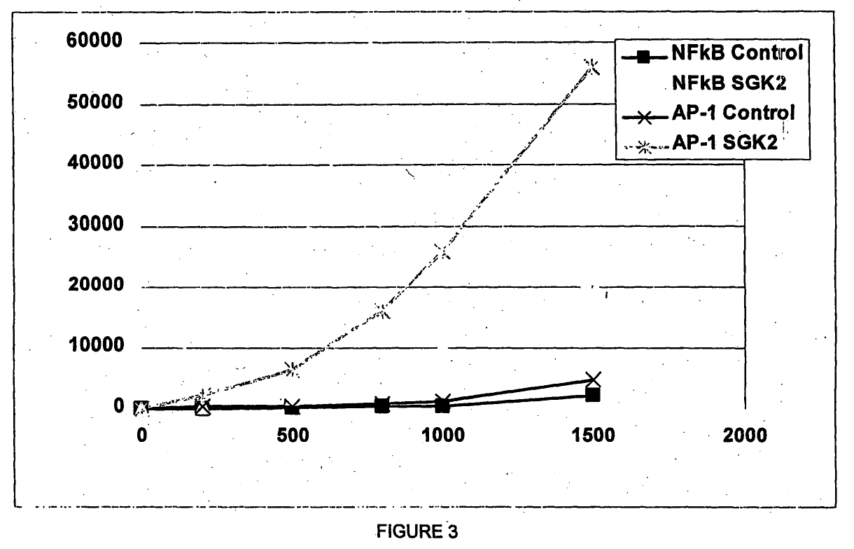

AP1 activity was measured in HEK293 cells and in HEK293 cells stably transfected with SGK2. SGK-37A clones have been shown to overexpress SGK2. AP1 activity was several fold higher in SGK-37A than in control HEK293 cells (FIG. 3). This data suggests that SGK2 can upregulate AP1 promoter activity in HEK293 cells. In the nucleus, AP1 transactivation induces the transcription of several genes involved in proliferation and several MMP genes. Our data suggests that SGK2 can induce an invasive phenotype via AP1 dependent upregulation of MMP gen expression.

SGK2 stimulates the translocation of beta catenin into the nucleus. SGK2 stabilizes beta catenin in HEK293 cells. To determine whether overexpression of SGK2 in HEK293 cells would induce beta catenin stability, we employed immunocytochemistry analysis. Monoclonal antibody for beta catenin was used in the analysis. In vivo expression of beta catenin was measured by standard protocols. The results indicate that SGK2 expressing cells have a higher concentration of beta catenin than parental cells. β catenin is localized entirely in the nucleus of SGK2 overexpressing cells, suggesting that SGK2 regulates the translocation of beta catenin into the nucleus.

Taken together, these results indicate that SGK2 is an important intracellular regulator of signaling via components of the Wnt/wingless pathway, specifically through modulation of GSK3β activity. Beta catenin has a consensus sequence phosphorylation site for GSK3β, and GSK3β acts to cause the degradation of beta catenin. Several studies have shown that GSK3β phosphorylates β catenin and that the phosphorylation of β catenin is essential for its degradation. If β catenin is not phosphorylated, the stability of β catenin increases in the cytoplasm and subsequently increases the translocation of beta catenin to the nucleus. In the nucleus, beta catenin associates with TCF4 to induce the transcription of several genes including cyclin D1.

SGK stimulates TCF4 transcriptional activity. The nuclear translocation of beta catenin is associated with a complex formation between β catenin and members of the high mobility group transcription factors, LEF1/TCF, which then activate transcription of target genes. LEF1 is a transcription factor that is by itself unable to stimulate transcription from multimerized sites, although in association with β catenin LEF1/TCF proteins can augment promoter activity from multimerized binding sites.

We examined the transcriptional activation of a synthetic TCF4/β catenin responsive promoter construct containing TCF4 binding sites in HEK293 cells overexpressing SGK2 and in control HEK293 cells. Higher promoter activity was observed only in SGK2 overexpressing cells. Transient transfection of increasing concentrations of TCF4 reporter gene produced concentration dependent TCF4 transactivation in SGK2 over expressing cells, whereas transient transfection of TCF4 reporter gene into HEK293 cells did not produce significant transactivation. This result indicates that SGK2 selectively targets GSK3β. Regulated β catenin subsequently increased the TCF4 transactivation in HEK293 cells. These data indicates that SGK2 overexpression overcomes the regulation of TCF4 expression by adhesion/deadhesion, and that it maintains constitutively high levels of TCF4 transactivation. TCF4/β catenin has been shown to induce transcription of genes encoding homeobox proteins that regulate mesenchymal genes, and this pathway is likely to mediate the epithelial to mesenchymal transformation. Constitutive activation of TCF/β catenin is oncogenic in human colon carcinomas. The data presented here show that SGK2 can modulate β catenin signaling and transactivate TCF4 reporter genes.

SGK2 stimulate NF-kB transcription. It has previously been shown that PKB/AKT regulate NF-κB mediated transactivation. In view of this, we next asked whether SGK2 could activate the NF-κB reporter assay in vivo. To evaluate NF-κB transactivation, the NF-κB promoter containing luciferase plasmid was transiently transfected into HEK293 cells overexpressing SGK2 and control HEK293 cells. As shown in FIG. 3, the activity of the NF-κB reporter was several fold higher in SGK2 overexpressing cells than in control HEK293 cells. Increasing concentration of NF-κB reporter plasmid in the SGK2 overexpressing cells increased luciferase activity, whereas NF-κB mediated transactivation had no significant effect on the control HEK293 cell. This data demonstrates that SGK2 can regulate NF-κB transactivation.

NF-kB transactivation occurs in response to the major proapoptotic signals, including TNF-α, anticancer drugs, and ionizing radiation. Several reports have indicated that in some cancer cell types, NF-κB is an important factor for cell survival. Hence, SGK2 may promote cell survival in certain cell types and participate in tumor promotion.

NF-kB DNA binding activity coincides with degradation of IκB alpha. To examine the status of IκB alpha in the SGK2 overexpressing cells, we performed the following experiment. Cellular extracts were made from HEK293 cells overexpressing SGK2 and control HEK293 cells. These cell extracts were analyzed against a specific anti-phospho IκB alpha antibody. Increasing concentrations of cell extract produced increasing IκKB alpha phospho signal, whereas the same protein concentration of control HEK293 cell extracts did not produce IκB alpha phospho signals. These results suggest that NF-κB activation by SGK2 is mediated by IκB alpha phosphorylation.

SGK2 phosphorylation of BAD. SGK2 phosphorylates some of the proteins phosphorylated by PKB. It has previously been shown that PKB can phosphorylate BAD. It was tested whether SGK2 phosphorylates BAD. Protein was isolated from HEK293 cells overexpressing SGK2 and control HEK293 cells; and the phosphorylation status of BAD was measured. The cells were lysed and the expression of BAD phosphorylation was determined by anti-BAD phospho antibody. SGK2 overexpressing cells contain higher levels of phospho Bad protein than normal cells, although expression levels of BAD protein were unaffected by SGK2. These finding show that SGK2 increases BAD phosphorylation in HEK293 cells.

Phosphorylation of BAD may lead to the prevention of cell death via a mechanism that involves the selective association of phosphorylated forms of BAD with 14-3-3 protein isoforms. The identification of BAD as a SGK2 substrate expands the list of in vivo SGK2 targets. Recent studies have revealed that BAD represents a point of convergence of several different signal transduction pathways that are activated by survival factors that inhibit apoptosis in mammalian cells. These data suggest that SGK2 inhibits apoptosis in mammalian cells through phosphorylation of BAD.

Phosphorylation of FKHR in HEK293 cells. The forkhead family of transcription factors is involved in tumorigenesis in rhabodomyosarcoma and acute leukemias. FKHR, FKHRL1, and AFX mediate signaling via a pathway involving IGFR1, PI3K and PKB/AKT. Phosphorylation of FKHR family members by PKB/AKT promotes cell survival and regulates FKHR nuclear translocation and target gene transcription. Insulin stimulation specifically promotes phosphorylation of this threonine site and causes FKHR cytoplasmic retention by 14-3-3 protein binding on the phosphorylated sequence.

To investigate whether FKHR could be phosphorylated by SGK2 in a cellular context, we created HEK293 cells stably expressing SGK2 and then examined FKHR phosphorylation with phospho specific antibodies. These experiments demonstrated that FKHR, Thr24 or Ser 256 were phosphorylated at low levels in normal HEK293 cells whereas HEK293 stable cells had higher levels of FKHR phosphorylation. This data shows that FKHR exhibits higher phosphorylation status in SGK2 overexpressing cells.

It has previously been known that FKHR phosphorylation leads to FKHR's interaction with 14-3-3 proteins and sequestration in the cytoplasm, away from its transcriptional targets. The unphosphorylated FKHR accumulates in the nucleus where it activates death genes, including Fas ligand gene, and thereby participates in the process of apoptosis. Upon phosphorylation, FKHR interacts with 14-3-3 and is retained in the cytoplasm thereby inhibiting its ability to activate transcription. Therefore, phosphorylation of FKHR by SGK2 can promote cell survival.

CREB phosphorylation is regulated by SGK2. To determine whether CREB is a regulatory target for SGK2, we performed the following experiments. Equal amounts of protein were isolated from SGK2 overexpressing cells as well as control HEK293 cells and subjected to phospho CREB analysis. The cells were lysed and the amount of CREB phosphorylation was determined by CREB phospho (Ser133) antibody. SGK2 overexpressing cells contain higher levels of phospho CREB protein than normal cells, showing that SGK2 increases CREB phosphorylation

Studies by have indicated that CREB function is important in promoting cell survival. Cyclin D1 expression is regulated by CREB. The majority of breast cancer cell lines and mammary tumors overexpress cyclin D1, suggesting that induction of cyclin D1 may play an important role in mammary tumorigenesis. These studies further clarify the mechanism by which SGK2 could promote cell survival. CREB function is important in promoting cell survival by responding to growth factor stimulation. These data imply that SGK2 modulates the phosphorylation status of CREB in vivo, and therefore is involved in cell survival through the CREB pathway.

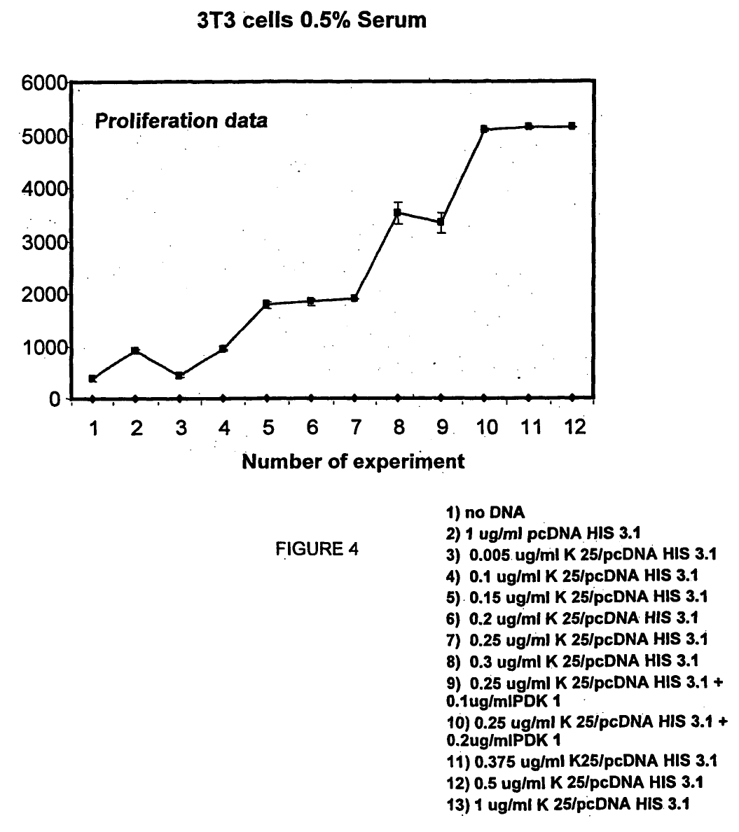

SGK2 is activated by PDK1 and the activation leads to increased kinase activity. To determine whether cloned and purified SGK2 can phosphorylate specific peptides directly, SGK2 was purified from insect cells. Activation was performed in vitro by mixing SGK2 and PDK1. After the activation, the PDK1 was removed from the mixture and purified SGK2 was used for the analysis. The cell extracts were purified by GST affinity column chromatography and the purity was analyzed by SDS-PAGE. Both non-activated and PDK1-activated SGK2 produced similar amounts of protein. SGK2 activated by PKD1 was significantly phosphorylated, while non-activated SGK2 was not. The data is shown in FIG. 4.

The kinase activity of SGK2 was evaluated using specific peptides. SGK2 was incubated with two different peptide substrates ((SEQ ID NO: 32) PKB-sub: CKRPRAASFAE; and (SEQ ID NO:33) PDK1: KTFCGTPEYLAPEV RREPRILS EEEQEMFRDFDYI (UBI Catalogue #12401), and in vitro kinase assays carried out. Equivalent concentration of purified SGK2 were incubated using a Beckman Biomek 2000 robotic system. Each well containing 25 μl reaction mixture composed of 10 μl SGK2, 5 μl of assay dilution buffer, 5 μl of peptide substrate and 5 μl of γ ³²P-ATP. The kinase reaction was carried out for 15 minutes at room temperature (22° C.). At the end of the reaction period, 10 μl of the reaction mixture was spotted onto 96-well p81 phosphocellulose multiscreen plates from Millipore, washed and the ³²P incorporation was counted in a Wallac Microbeta scintillation counter.

Peptides incubated with purified SGK 2 gave significant incorporation of ³²P, whereas in the absence of peptides no significant incorporation was seen. When comparing the peptides, PKB-sub had significant incorporation of ³²P whereas addition of same amount of control peptide (PDK1 peptide) had no significant incorporation. This data demonstrates that purified SGK2 possesses a kinase activity in vitro. Moreover, the PDK1 activated SGK2 had significantly higher kinase activity compared to non-activated SGK2. These data clearly demonstrate that activated SGK2 phosphorylates the GSK3 Ser9 (GSK3β consensus) sequence, supporting the previous observation that SGK2 overexpressing cells exhibit higher level of GSK3 Ser9 phosphorylation than control cells.

SGK2 kinase activity is stimulated by Calyculin A and Okadaic acid. Hi5 insect cells expressing GST-SGK2 were treated with 100 nM microcysteine, 99.8 nM okadaic acid and 49.8 nM calyculin A for four hours at 27° C. The GST-SGK2a fusion protein was purified by GST-agarose affinity column and eluted with 20 mM Glutathione/50 mM Tris-HCl/50 mM NaCl, pH 7.5. Substrates were PKB sub and CapK sub at 1 mg/ml, for 15 minutes at room temperature. The results were as follows:

| | No-Substrate | PKB sub | CapK sub |
|---|---|---|---|
| | (CCPM1) | (CCPM1) | (CCPM1) |
| Untreated | 349 | 979 | 1081 |
| Microcysteine | 305 | 217 | 330 |
| Calyculin A | 0 | 92540 | 59335 |
| Okadaic Acid | 2078 | 132171 | 161553 |

These data indicate that okadaic acid and Calyculin A stimulated SGK2 kinase activity, suggesting that okadaic and Calyculin A can stimulate SGK2 activity. It has previously been shown that protein phosphatase inhibitors such as okadaic acid and Calyculin A modulate phosphorylation of several nuclear proteins.

These findings demonstrate SGK2 could promote cell survival and cell growth. Overexpression of SGK2 in HEK293 cells increased GSK3 phosphorylation thereby inhibiting the activity of GSK3, and subsequently leading to AP1 transactivation. GSK3 is involved in regulation of several intracellular signaling pathways, of which the Wnt pathway is of particular interest. In mammals, Wnt signaling increases the stability of beta catenin resulting in transcriptional activation of LEF-1/TCF. In SGK2 overexpressing cells we have shown increased LEF-1/TCF transactivation through increasing the stability of the beta catenin pool in the cells, suggesting that SGK2 activates the Wnt signaling pathway, which can lead to nuclear localization of beta catenin and increased transactivation of LEF-1/TCF.

At least 6 SGK2 substrates have been identified in mammalian cells, and they fall into two main classes: regulators of apoptosis and regulators of cell growth, including protein synthesis and glycogen metabolis. The SGK2 substrates involved in cell/death regulation include Forkhead transcription factors (FKHR), the pro-apoptotic Bcl-2 family member BAD, and the cyclic AMP response element binding protein (CREB).

We have also demonstrated that SGK2 could regulate signaling pathways that lead to induction of the NF-κB family of transcription factors in HEK293 cells. This induction occurs at the level of degradation of the NF-κB inhibitor IκB and is specific for NF-κB. These data suggest that SGK2 appears to be a point of convergence for several different signaling pathways. Taken together, our results suggest that the over expression of SGK2 may therefore play a central role in tumor cell progression.

Materials and Methods.

Buffers, reagents and methods were as described in Example 2, unless otherwise stated.

Cloning of full length SGK2. To generate the full length cDNA of SGK2, a pair of primers were designed and used in a PCR reaction. The amplification product was cloned through restriction sites, EcoR I and Xho I, into bacteria expression vector pGEX-4T-3 and mammalian expression vector pcDNA3.1/His B. All construct were verified by restriction enzyme digestion and DNA sequencing.

Expression and Purification of SGK2 Protein. The human SGK2 gene was subcloned into baculovirus transfer vector pAcG2T (BD PharMingen) under the control of the strong AcNPV (Autograpga californica Nuclear Polyhedrosis Virus) polyhedrin promoter. This was co-transfected with linear BaculoGoldTM DNA in Spodoptera frugiperda Sf9 cells following the manufacturer's procedure (BD PharMingen). The high titer of GST-SGK2 recombinant baculovirus was amplified in Sf9 cells in TNM-FH medium (JHR Biosciences) with 10% fetal bovine serum. The GST-SGK2 protein was expressed in about 5×10⁸ Hi5 cells (Invitrogen) in 500 ml of Excell-400 medium (JHR Biosciences) with about 5 MOI for a period of 72 h in a spinner flask. The cells were harvested at 800×g for 5 min at 4° C. The pellet was lysed in 40 ml of Lysis Buffer by sonication and centrifuged at 10,000×g at 4° C. for 15 min. The supematant was loaded on the column contained 2.5 ml of glutathione-agarose (Sigma). The column was washed with Wash Buffer A until OD280 returned to baseline, then Wash Buffer B. The GST-SGK2 protein was eluted in Elution Buffer. The fraction was aliquoted and stored at −70° C.

Assay of SGK2. SGK2 was assayed at room temperature for 15 min with 25 μl of reaction mixture containing 5 mM MOPS, PH7.2, 5 mM MgCl₂, 5 mM β-glycerophosphate, 50 μM dithiothreitol, 1 μM β-methyl aspartic acid, 1 mM EGTA, 0.5 mM EDTA, 0.5 μM PKI, 50 μM [γ-³²P]-ATP and 0.2 μg/ul PKB-sub peptide (UBI) or PDKtide peptide (UBI) as substrates. GSK3 consensus peptide (SEQ ID NO:34, PKB-sub: CKRPRAASFAE), PDK1 sub-SEQ ID NO:35, KTFCGTPEYLAPEVRREPRILSEEEQEMFRDFDYI. Reactions were initiated by addition of [γ-³²P]-ATP and terminated by spotting 10 μl of aliquots onto cellulose phosphate paper in 96-well filtration plate (Millipore), followed by washing in 1% phosphoric acid. The dried plate was added 25 μl scintillant (Amersham) and counted.

SGK2 Phosphorylation by PDK1. SGK2 was incubated with active His-tag PDK1 in the presence of Mg²⁺/ATP. His-tag PDK1 was expressed in insect cells and purified on Talon affinity column. SGK2 phosphorylation assay was performed at room temperature for 20 min in 25 μl of reaction solution consisting of 10 mM MOPS, PH 7.2, 15 mM MgCl₂, 5 mM β-glycerophosphate, 1 mM EGTA, 0.2 mM sodium orthovanadate, 0.2 mM dithiothreitol, 0.5 μM PKI, 0.2 μM Microcystin-LR, 75 ng/μl Ptdlns (3, 4, 5) P3 (PIP3), 156 ng/μl dioleoyl phosphatidylcholine (DOPC), 156 ng/μl dioleoyl phosphatidylserine (DOPS), 50 μM [γ-³²P]-ATP, ˜20 ng His-PDK1 and ˜5 μg GST-SGK2. The reaction were incubated and terminated by addition of 25 μl 2× loading buffer. No PDK1 was added to negative control reaction. 25 μl of above loading samples were run on 9% SDS-PAGE. The dried Coomassia blue-stained gel was imaged in GS-525 Molecular Imagera System (BIO-RAD).

SGK2 Activation by PDK1. About 2.5 mg of GST-SGK2 and 1 μg of His-PDK1 were incubated at 4° C. for 2 hours in 20 ml of activation solution containing 10 mM MOPS, PH 7.2, 15 mM MgCl₂, 5 mM β-glycerophosphate, 1 mM EGTA, 0.2 mM sodium orthovanadate, 0.2 mM dithiothreitol, 0.5 μM PKI, 0.2 μM Microcystin-LR, 75 ng/μl Ptdlns (3, 4, 5) P3 (PIP3), 156 ng/μl dioleoyl phosphatidylcholine (DOPC), 156 ng/μl dioleoyl phosphatidylserine (DOPS), and 10 mM ATP. The glutathione was removed from the activation solution on Q-sepharose column. The activated GST-SGK2 were re-purified from glutathione-agarose column.

Cell and cell culture. 293 cells were stably transfected with a mammalian vector incorporating SGK2 to produce overexpressing wild type SGK2. Cells were grown in MEM containing 10% FCS, 2 mm L-glutamine, glucose (3.6 mg/ml), insulin (10 μg/ml), and G418 (40 μg/ul) were added to transfected cells to maintain selection pressure.

Transient transfection: HEK293 cells were seeded at 1.5×10⁵ cells/well plate and grown for 24 hr before transfection. Various concentration of plasmid DNA were transfected using Fugen (Roche) according to the manufucture's protocol. DNA content was normalized with appropriate empty expression vectors. Cells were starved for O/N in DMEM containing 0.5% FBS.

Western blotting: Cells were lysed for 10 minutes on ice in NP-40 lysis buffer (1% NP40, 50 mM Hepes, pH 7.4, 150 mM Nacl, 2 mM EDTD, 2 mM PMSF, 1 mM Na-o-vanadate, 1 mM NaF, 10 μg/ml aprotinin, 10 μg/ml leupeptin). Extracts were centrifuged with the resulting supernatants being the cell lysate used in assays. Lysates were electrophoresed through SDS-PAGE and transferred to immobilin-P (Millipore Bedford, Md.). Antibodies used to probe Western blots were: Anti-Xpresss, Phospho-FKHR (Thr24, Caspase-9, Phospho-IkBalpha (Ser32/36), Bad, Phospho CREB, Phospho GSK3 alpha (ser-9), GSK3 monoclonal, (New England Biolab, Mississauga, ON, Canada) Bands were visualized with ECL chemiluminescent substrate (Amersham Pharmacia biotech).

Reporter assay: 293 cells were transfected in 6-well plates with Fugene (Roche Diagnostics) according to the manufacture's instructions. To analyse various reporter assay, respective reporter construct were transiently transfected with indicated amount of luciferase reporter gene construct series of LEF-1/TCF binding sites, AP1 binding sites and NF-κB binding sites. Extracts were prepared and assayed 24-48 after transfection and relative luciferase activity was determined using Promega Dual luciferase reporter assay system as described by the manufacture.

Immunocytochemistry: 293-cell lines were grown in 8 chamber slides for 2 days, washed with PBS, fixed in absolute cold methanol for 10 minutes, washed with PBS and incubated overnight at 40° C. with beta-catenin (#C19220-BD Transduction Laboratories), His-Prob (#Sc-803, Santa Cruz, USA) and anti-Xpress antibody (R910-25, Invitrogen), all diluted 1:100 in PBS with 0.1% Triton X-100, then washed with PBS. Proceed with immunostaining by using the ABC method (ABC-Elite kit, Vector). Acccording to the amount and intensity of staining, the scale was divided into 2 classes. The slides designated “+” had positive staining intensity, slides designated “−” showed no immunoreactivity. In addition to conventional light microscopic examination, in order to quantitate the amount of reactivity, specimens were also investigated by computerized image analysis using image pro (Media Cybernetics, Md., USA).

Expression and Purification of GST-SGK2a from Hi 5 insect cells Human SGK2a was cloned into the Baculovirus vector pAcG2T with the multiple cloning sites in the vector. This vector contains an N-terminal Glutathione S-transferase tag (GST-tag) which allows for affinity purification on Glutathione agarose beads. The vector was infected into Sf9 insect cells via lipid vesicles. The titer of the baculovirus particles was amplified in Sf9 insect cells. The amplified baculovirus titer was then used to infect four 250 ml volume spinner-flasks (Pyrex) containing Hi 5 cells which were at approximately 0.8×10⁶ cells/ml. The expression of the fusion protein cells were incubated at 27° C., with spinning at 80 rpm, over 3.5 days. Near the end of this expression period, each of the four 180 ml cultures of Hi 5 cells were stimulated with a 4 hour, 27° C. treatment with either 100% DMSO (negative control) or one of three different PP1 and PP2a phosphatase inhibitors: 100 nM Microcystin (Calbiochem), 55.05 nM Calyculin A (Calbiochem), and 96.9 nM Okadaic Acid (Calbiochem). Finally, the cells were collected by centrifugation in Beckman Avant-25 rotor ID 10.500 at 3000 rpm, 5 min, 4° C. After a brief 1×PBS wash, the cells were resuspended in a 50 mM Tris-HCl/1% NP-40, pH 7.5 lysis buffer supplemented with the following protease inhibitors: 100 μM Sodium Vanadate, 1 mM glycerophosphate, and 237 μl Protease Inhibitor Cocktail Set III (Calbiochem). The cells were lysed using the small probe of the sonic dismembrator: output 1:3 repititions of 8 sec on and 5 sec pause. Once the cytosolic proteins are released into the supernatant, the cellular debris is removed by centrifugation in Beckman Avanti-30: 14,000 rpm, 15 min, 4° C. The lysate supernatant is applied to Glutathione-agarose beads (SIGMA) and allowed to batch-bind, rotating end-over-end, at 4° C. for 30 mins. Non-specific proteins are washed from the beads 5 times with STEL 500 (50 mM Tris-HCl/500 mM NaCl, pH 7.5) followed by 5 times with STEL 50 (50 mM Tris-HCl/50 mM NaCl, pH 7.5). Finally, the GST-tagged fusion protein is eluted from the beads with Elution buffer (20 mM glutathione/50 mM Tris-HCl/50 mM NaCl). Purified SGK2a kinase activity is assayed against PKB peptide SEQ ID NO:36 (CKRPRAASFAE), a universal SRC kinase family substrat and CapK peptide SEQ ID NO:37 (CGRTGRRNSI).

### EXAMPLE 4

GRK5

Genbank sequences were screened as described in Example 1. Analysis of BLASTN and BLASTX outputs identified a EST sequence from IMAGE clone Al358974 that had potential for being associated with a sequence encoding a kinase domain-related protein, e.g., the sequence had homology, but not identity, to known kinase domain-related proteins.

The Al358974 IMAGE clone was sequenced using standard ABI dye-primer and dye-terminator chemistry on a 377 automatic DNA sequencer. Sequencing revealed that the sequence corresponds to SEQ ID NO:7. SEQ ID NO:20 and 21 were used for amplification.

The expression of GRK5 was determined dot blot analysis, and the protein was found to be upregulated in several tumor samples.

Dot blot preparation. Total RNA was purified from clinical cancer and control samples taken from the same patient. Samples were used from both liver and colon cancer samples. Using reverse transcriptase, cDNAs were synthesized from these RNAs. Radiolabeled cDNA was synthesized using Strip-EZ™ kit (Ambion, Austin, Tex.) according to the manufacturer's instructions. These labeled, amplified cDNAs were then used as a probe, to hybridize to human protein kinase arrays comprising human GRK5. The amount of radiolabeled probe hybridized to each arrayed EST clone was detected using phosphorimaging.

The expression of GRK5 was substantially upregulated in the tumor tissues that were tested. The data is shown in Table 6, expressed at the fold increase over the control non-tumor sample.

TABLE 6

| | liver | liver | liver | colon | colon | colon | colon | colon | colon | colon |
|---|---|---|---|---|---|---|---|---|---|---|
| | 1 | 2 | 3 | 1 | 4 | 5 | 7 | 8 | 9 | 10 |
| GRK5 | 1.5 | 0.7 | 2.6 | 1.8 | 1.3 | 4.3 | 1.9 | 0.4 | 0.7 | 2.00 |
| beta-actin | 2.05 | 1.07 | 1.57 | 0.42 | 1.28 | 2.19 | 1.20 | 4.60 | 0.60 | 0.49 |
| GAPDH | 1.30 | 0.33 | 1.25 | 0.76 | Not | Not | Not | Not | Not | Not |
| | | | | | done | done | done | done | done | done |
| K413 | Not | Not | Not | Not | 1.72 | 2.36 | 2.10 | 1.00 | 1.00 | 1.68 |
| (ribosomal | done | done | Done | Done | | | | | | |
| protein) | | | | | | | | | | |

Expression of GRK5. To characterize GRK5 at the protein level, Hi5 cells were transfected with pAcG4T3-GRK5. The ORF was cloned into baculovirus expression vector pAcG2T (BD pharmagen). This construct construct was then co-transfected with linear BaculoGold DNA into Sf9 cells to obtain an isolated recombinant virus. The recombinant virus was amplified and then used to infect Sf9 cells. GRK5 expressed in Hi5 cells was purified by glutathione-sepharose column chromatography. Cell lysates were prepared from these cell lines for further analysis. Briefly, the precipitations were performed with ectopically expressed tagged GRK5 from insects cells as described in the method section. This will enable us to perform in vitro kinase assays for the identification of specific inhibitors of this kinase.

To characterize GRK5 at the protein level, HEK293 cells were transfected with pcDNA3-X-press-GRK5 by standard methods. The transiently transfected cell lines were used to prepare whole cell lysates which were analysed by Western blotting with an anti-X-press mmonoclonal antibody. These experiments revealed a fusion protein in the stably transfected cell lines, whereas HEK293 cell lines transfected with the vector only control did not have this protein. Similarly, we also detected GRK5 in transfected Hi5 cells.

The anti-X-press antibody was used to purify the kinase via immunoprecipitation. Anti-X-press antibody precipitated fusion protein was subjected to SDS-PAGE analysis. SDS-PAGE indicated that we could successfully purify the GRK5 from the lysates from transfected cells.

Next, anti-X-press antibody immunoprecipitated materials and glutathione-sepharose chromatography purified materials were used for in vitro kinase assays. Casein, MBP and phosvitin were found to be phosphorylated by purified GRK5. In the absence of substrate there was no significant incorporation of radioactive materials (³⁵P) indicating that GRK5 does not autophosphorylate under these conditions. This suggests that glutathione-sepharose and X-press antibody purified materials possess a kinase activity and that this kinase activity is capable of phosphorylating substrates in vitro.

Expression and Purification of GRK5 Protein. The human GRK5 gene was subcloned into baculovirus transfer vector pAcG4T3 derived from pAcG2T (BD Biosciences) under the control of the strong AcNPV (Autograpga californica Nuclear Polyhedrosis Virus) polyhedrin promoter. This was co-transfected with linear BaculoGold DNA in Spodoptera frugiperda Sf9 cells using standard techniques (BD Biosciences). The GST-GRK5 recombinant baculovirus was amplified in Sf9 cells in TNM-FH medium (JHR Biosciences) with 10% fetal bovine serum. The GST-GRK5 protein was expressed in about 5×10⁸ Hi5 cells (Invitrogen) in 500 ml of Excell-400 medium (JHR Biosciences) at a multiplicity of infection (MOI) of five for 72 h in a spinner flask. The cells were harvested at 800×g for 5 min at 4° C. The pellet was lysed in 40 ml of Lysis Buffer by sonication and centrifuged at 10,000×g at 4° C. for 15 min. The supernatant was loaded onto a column containing 2.5 ml of glutathione-sepharose (Sigma). The column was washed with Wash Buffer A until OD280 returned to baseline. The column was then washed with Wash Buffer B. The GST-GRK5 protein was eluted in Elution Buffer. The eluted protein was aliquoted and stored at −70° C.

### EXAMPLE 5

DM-PK

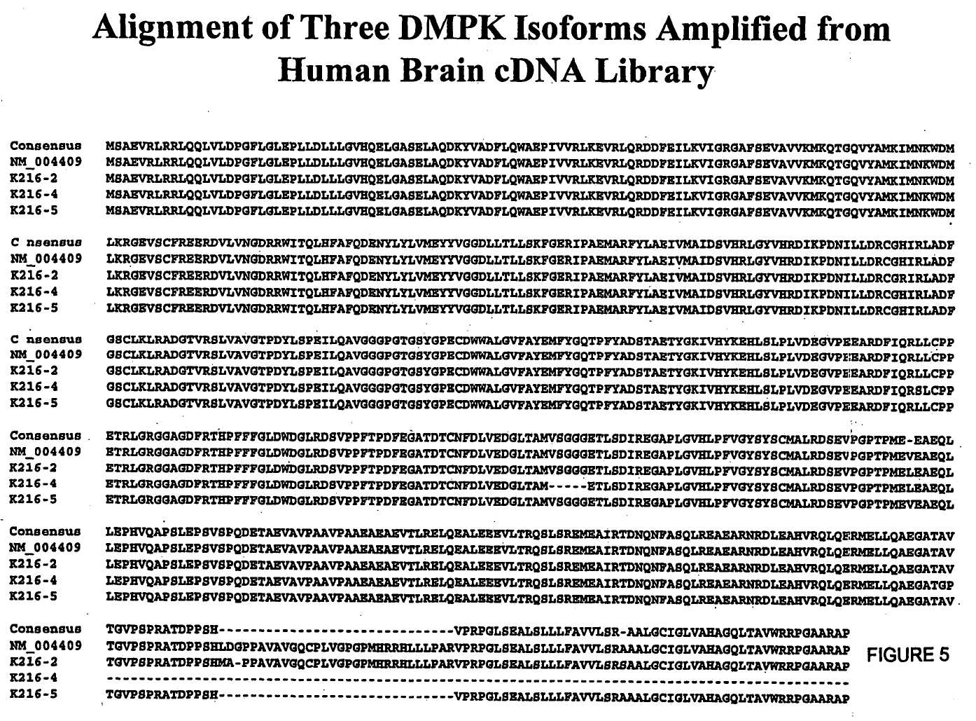

The Genbank EST database was searched as described in Example 1. Analysis of the BLASTN and BLASTX outputs identified a EST sequence from IMAGE clone Al886007 that had potential for being associated with a sequence encoding a kinase domain-related protein, e.g., the sequence had homology, but not identity, to known kinase domain-related proteins. The Al886007 IMAGE clone was sequenced using standard ABI dye-primer and dye-terminator chemistry on a 377 automatic DNA sequencer. Sequencing revealed that the sequence corresponds to SEQ ID NO:9. SEQ ID NO:22 and 23 were used for amplification. The expression of DM-PK was determined dot blot analysis, and the protein was found to be upregulated in several tumor samples. As shown in FIG. 5, a number of isoforms of DMPK were characterized, including SEQ ID NO:10; SEQ ID NO:38 and SEQ ID NO:39.

Dot blot preparation. Total RNA was purified from clinical cancer and control samples taken from the same patient. Samples were used from both liver and colon cancer samples. Using reverse transcriptase, cDNAs were synthesized from these RNAs. Radiolabeled cDNA was synthesized using Strip-EZ™ kit (Ambion, Austin, Tex.) according to the manufacturer's instructions. These labeled, amplified cDNAs were then used as a probe, to hybridize to human protein kinase arrays comprising human DM-PK. The amount of radiolabeled probe hybridized to each arrayed EST clone was detected using phosphorimaging.

The expression of DM-PK was substantially upregulated in the tumor tissues that were tested. The data is shown in Table 7, expressed at the fold increase over the control non-tumor sample.

TABLE 7

| | liver | liver | liver | colon | colon | colon | colon | colon | colon | colon |
|---|---|---|---|---|---|---|---|---|---|---|
| | 1 | 2 | 3 | 1 | 4 | 5 | 7 | 8 | 9 | 10 |
| DM-PK | 1.8 | 1.2 | 2.8 | 2 | 2.0 | 1.7 | 4.5 | 0.9 | 1.2 | 2.35 |
| beta-actin | 2.05 | 1.07 | 1.57 | 0.42 | 1.28 | 2.19 | 1.20 | 4.60 | 0.60 | 0.49 |
| GAPDH | 1.30 | 0.33 | 1.25 | 0.76 | Not | Not | Not | Not | Not | Not |
| | | | | | done | done | done | done | done | done |
| K413 | Not | Not | Not | Not | 1.72 | 2.36 | 2.10 | 1.00 | 1.00 | 1.68 |
| (ribosomal | done | done | done | done | | | | | | |
| protein) | | | | | | | | | | |

The data displayed in Table 8 provides a brief summary of the pathology report of the patient samples.

TABLE 8

| | | | | | | | Lym- | |
|---|---|---|---|---|---|---|---|---|
| | | | | Site of | | Vascu- | phatic | |
| | | | Precursor | Involve- | | lar Inva- | Involve- | Meta- |
| Patient | Age | Gender | Adenoma | ment | Differentiation | sion | ment | stasis |
| Liver | 49 | Female | N/a | Liver | Moderately | No | Yes | No |
| 1 | | | | | Differentiated | | | |
| Liver | 53 | Male | N/a | Liver | Moderately | Yes | No | No |
| 2 | | | | | Differentiated | | | |
| Liver | 75 | Female | Yes | Right | Moderately | No | No | No |
| 3 | | | | Colon | differentiated | | | |
| Colon | 55 | Female | No | Rectum | Moderately | N/A | Yes | No |
| 1 | | | | | Differentiated | | | |
| Colon | 91 | Female | Yes | Cecum | Moderately | No | Yes | No |
| 4 | | | | | Differentiated | | | |
| Colon | 79 | Male | No | Ileum | Moderately | No | No | No |
| 5 | | | | and | Differentiated | | | |
| | | | | Colon | | | | |
| Colon | 93 | Male | No | Recto- | Moderately | No | No | No |
| 7 | | | | sigmoid | Differentiated | | | |
| Colon | 61 | Male | Yes | Yes | Moderately | No | Yes | Yes, |
| 8 | | | | | Differentiated | | | Liver |
| Colon | 60 | Male | No | Recto- | Moderately | Yes | No | Yes, |
| 9 | | | | Sigmoid | Differentiated | | | Liver |
| Colon | 60 | Male | No | Sigmoid | Moderately | Yes | Yes | No |
| 10 | | | | Colon | Differentiated | | | |

Expression of DM-PK in E. coli. To characterize DM-PK at the protein level, E. coli cells were transformed with pGEX-DM-PK. The DM-PK ORF was cloned into a pGEX vector (Pharmacia) that was used to transform E. coli. A transformed colony was selected and cultured in order to express the GST-DM-PK fusion protein. The fusion protein was purified via glutathione-sepharose column chromatography. The purified fraction was analysed by SDS-PAGE, and showed a band corresponding to the DM-PK protein.

As an alternative expression system, we transfected HEK293 cells with DM-PK. Cell lysates of the transfected cells were prepared. We utilized an anti-X-press antibody to immunoprecipitate the recombinant DM-PK. This data shows successful expression and purification of DM-PK from transfected HEK293 cells.

Kinase Activity. DM-PK purified from both E. coli and transfected HEK293 was used for in vitro kinase assays. MBP and Histone H1 were both phosphorylated by purified DM-PK in these assays. In the absence of added substrate, there was no significant incorporation of radioactive materials (³²P) indicating that DM-PK does not autophosphorylate under these conditions. This data shows that purified DM-PK possesses kinase activity.

Experimental procedures. DM-PK was subcloned into bacterial expression vector pGEX-4T3 (Pharmacia) using EcoR1 and Not I sites. The GST-DM-PK protein was produced in E. coli DH5a cells in 2×YT media in 150 uM IPTG at 37° C. overnight. The cells were harvested at 10,000×g for 10 minutes at 4° C. The pellet was suspended in 50 ml of Lysis Buffer (150 mM Tris-Hcl pH 7.5, 2.5 mM EDTA, 150 mM Mg Cl₂, 1% NP-40, 0.1% β-mercaptoethanol, 0.1 mM PMSF, 1 mM benzamide and 10 μl trypsin inhibitor), sonicated, and centrifuged at 10,000×g for 15 minutes at 4° C. The supernatant was loaded onto a 3 ml glutathione-sepharose column. The column was washed by wash buffer (50 mM Tris-Hcl, pH 7.5, 1 mM EDTA, 500 mM Nacl, 0.1% β-mercaptoethanol, 0.1% NP-40, 0.1 mM PMSF and 1 mM benzamide) and eluted with standard elution buffer.

### EXAMPLE 6

PDK2 Sequence

The Genbank database was searched for ESTs showing similarity to known kinase domain-related proteins as described in Example 1. Analysis of the BLASTN and BLASTX outputs identified a EST sequence from IMAGE clone Af309082 that had potential for being associated with a sequence encoding a kinase domain-related protein, e.g., the sequence had homology, but not identity, to known kinase domain-related proteins. The Af309082 IMAGE clone was sequenced using standard ABI dye-primer and dye-terminator chemistry on a 377 automatic DNA sequencer. Sequencing revealed that the sequence corresponds to SEQ ID NO:11; and a second sequence corresponds to SEQ ID NO:13.

Total RNA was purified from clinical cancer and control samples, and cDNAs synthesized by reverse transcriptase. cDNA corresponding to normal and tumor tissue from the same set were simultaneously amplified and labeled with alpha dCTP. Labeled, amplified cDNAs were then used to hybridize to human protein kinase arrays containing 354 protein kinases. The amount of radiolabeled probe hybridizing to each arrayed EST clone was detected using phosphorimaging. Through this process it was determined the PDK2 was upregulated in both colon and liver tumor tissue as compared to matched control tissue.

What is claimed is:

1. A method of screening for biologically active agents that modulate a cancer associated protein kinase function, the method comprising: combining a candidate biologically active agent with any one of:
(a) a polypeptide encoded by any one of SEQ ID NO:1, 3, 5, 7, 9, 11 or 13; or having the amino acid sequence set forth in SEQ ID NO:38 or SEQ ID NO:39;
(b) a cell comprising a nucleic acid encoding a polypeptide encoded by any one of SEQ ID NO:1, 3, 5, 7, 9, 11 or 13; or
(c) a non-human transgenic animal model for cancer associated kinase gene function comprising one of: (i) a knockout of a gene corresponding any one of SEQ ID NO:1, 3, 5, 7, 9, 11 or 13; (ii) an exogenous and stably transmitted mammalian gene sequence comprising polypeptide encoded by any one of SEQ ID NO:1, 3, 5, 7, 9, 11 or 13; and
determining the effect of said agent on kinase function.

2. A method for the diagnosis of cancer, the method comprising:
determining the upregulation of expression in any one of SEQ ID NO:1, 3, 5, 7, 9, 11, 13, 38 or 39 in said cancer.

3. The method of claim 2, wherein said cancer is a liver cancer.

4. The method of claim 2, wherein said cancer is a colon cancer.

5. The method of claim 2, wherein said determining comprises detecting the presence of increased amounts of mRNA in said cancer.

6. The method of claim 2, wherein said determining comprises detecting the presence of increased amounts of protein in said cancer.

7. A method for inhibiting the growth of a cancer cell, the method comprising downregulating activity of the polypeptide encoded by any one of SEQ ID NO:1, 3, 5, 7, 9, 11 or 13 or having the aminoacid sequence set forth in SEQ ID NO:38 or SEQ ID NO:39; in said cancer cell.

8. The method according to claim 7, wherein said method comprises introducing antisense sequences specific for any one of SEQ ID NO:1, 3, 5, 7, 9, 11 or 13.

9. The method according to claim 7, wherein said method comprises introducing an inhibitor of kinase activity into said cancer cell.

10. The method according to claim 7, wherein said cancer cell is a liver cancer cell.

11. The method according to claim 7, wherein said cancer cell is a colon cancer cell.

12. A method of screening for targets of a cancer associated protein kinase, wherein said targets are associated with signal transduction in cancer cells, the method comprising:
comparing the pattern of gene expression in a normal cell, and in a tumor cell characterized by up-regulation of any one of SEQ ID NO:1, 3, 5, 7, 9, 11, 13, 38 or 39.

13. The method according to claim 12, wherein said comparing the pattern of gene expression comprises quantitating specific mRNAs by hybridization to an array of polynucleotide probes.

14. A method of screening for targets of a cancer associated protein kinase, wherein said targets are associated with signal transduction in cancer cells, the method comprising:
comparing the pattern of protein phosphorylation in a normal cell, and in a tumor cell characterized by up-regulation of any one of SEQ ID NO:1, 3, 5, 7, 9, 11, 13, 38 or 39.

15. The method according to claim 12 or claim 14, wherein said signal transduction involves activation by protein dependent kinase 1.

16. An isolated nucleic acid comprising the sequence set forth in any one of SEQ ID NO:1, 3, 5, 7, 9, 11 or 13.


SEQUENCE LISTING (39 sequences)

SEQ ID NO: 1 - DNA, 3558 bp, Homo sapien
  CDS (482)...(3023)
ggaagaagggagcggggtcggagccgtcggggccaaaggagacggggccaggaacaggcagtctcggcccaactgcggacgctccctccaccccctgcgcaaaaagacccaaccggagttgaggcgctgcccctgaaggccccaccttacacttggcgggggccggagccaggctcccaggactgctccagaaccgagggaagctcgggtccctccaagctagccatggtgaggcgccggaggccccggggccccacccccccggcctgaccacactgccctgggtgccctcctccagaagcccgagatgcggggggccgggagacaacactcctggctccccagagaggcgtgggtctggggctgagggccagggcccggatgcccaggttccgggactagggccttggcagccagcgggggtggggaccacgggcacccagagaaggtcctccacacatcccagcgccggctcccggccatggagcccttgaagagcctcttcctcaagagccctctagggtcatggaatggcagtggcagcgggggtggtgggggcggtggaggaggccggcctgaggggtctccaaaggcagcgggttatgccaacccggtgtggacagccctgttcgactacgagcccagtgggcaggatgagctggccctgaggaagggtgaccgtgtggaggtgctgtcccgggacgcagccatctcaggagacgagggctggtgggcgggccaggtgggtggccaggtgggcatcttcccgtccaactatgtgtctcggggtggcggcccgcccccctgcgaggtggccagcttccaggagctgcggctggaggaggtgatcggcattggaggctttggcaaggtgtacaggggcagctggcgaggtgagctggtggctgtgaaggcagctcgccaggaccccgatgaggacatcagtgtgacagccgagagcgttcgccaggaggcccggctcttcgccatgctggcacaccccaacatcattgccctcaaggctgtgtgcctggaggagcccaacctgtgcctggtgatggagtatgcagccggtgggcccctcagccgagctctggccgggcggcgcgtgcctccccatgtgctggtcaactgggctgtgcagattgcccgtgggatgcactacctgcactgcgaggccctggtgcccgtcatccaccgtgatctcaagtccaacaacattttgctgctgcagcccattgagagtgacgacatggagcacaagaccctgaagatcaccgactttggcctggcccgagagtggcacaaaaccacacaaatgagtgccgcgggcacctacgcctggatggctcctgaggttatcaaggcctccaccttctctaagggcagtgacgtctggagttttggggtgctgctgtgggaactgctgaccggggaggtgccataccgtggcattgactgccttgctgtggcctatggcgtagctgttaacaagctcacactgcccatcccatccacctgccccgagcccttcgcacagcttatggccgactgctgggcgcaggacccccaccgcaggcccgacttcgcctccatcctgcagcagttggaggcgctggaggcacaggtcctacgggaaatgccgcgggactccttccattccatgcaggaaggctggaagcgcgagatccagggtctcttcgacgagctgcgagccaaggaaaaggaactactgagccgcgaggaggagctgacgcgagcggcgcgcgagcagcggtcacaggcggagcagctgcggcggcgcgagcacctgctggcccagtgggagctagaggtgttcgagcgcgagctgacgctgctgctgcagcaggtggaccgcgagcgaccgcacgtgcgccgccgccgcgggacattcaagcgcagcaagctccgggcgcgcgacggcggcgagcgtatcagcatgccactcgacttcaagcaccgcatcaccgtgcaggcctcacccggccttgaccggaggagaaacgtcttcgaggtcgggcctggggattcgcccacctttccccggttccgagccatccagttggagcctgcagagccaggccaggcatggggccgccagtccccccgacgtctggaggactcaagcaatggagagcggcgagcatgctgggcttggggtcccagttcccccaagcctggggaagcccagaatgggaggagaaggtcccgcatggacgaagccacatggtacctggattcagatgactcatcccccttaggatctccttccacacccccagcactcaatggtaaccccccgcggcctagcctggagcccgaggagcccaagaggcctgtccccgcagagcgcggtagcagctctgggacgcccaagctgatccagcgggcgctgctgcgcggcaccgccctgctcgcctcgctgggccttggccgcgacctgcagccgccgggaggcccaggacgcgagcgcggggagtccccgacaacaccccccacgccaacgcccgcgccctgcccgaccgagccgcccccttccccgctcatctgcttctcgctcaagacgcccgactccccgcccactcctgcacccctgttgctggacctgggtatccctgtgggccagcggtcagccaagagcccccgacgtgaggaggagccccgcggaggcactgtctcacccccaccggggacatcacgctctgctcctggcaccccaggcaccccacgttcaccacccctgggcctcatcagccgacctcggccctcgccccttcgcagccgcattgatccctggagctttgtgtcagctgggccacggccttctcccctgccatcaccacagcctgcaccccgccgagcaccctggaccttgttcccggactcagaccccttctgggactccccacctgccaaccccttccaggggggcccccaggactgcagggcacagaccaaagacatgggtgcccaggccccgtgggtgccggaagcggggccttgagtgggccaggccactcccccgagctccagctgccttaggaggagtcacagcatacactggaacaggagctgggtcagcctctgcagctgcctcagtttccccagggaccccacccccctttgggggtcaggaacactacactgcacaggaagccttcacactggaagggggacctgcgcccccacatctgaaacctgtaggtccccccagctcacctgccctactggggcccaacactgtacccagctggttgggaggaccagagcctgtctcagggaattgcctgctggggtgatgcagggaggaggggaggtgcagggaagaggggccggcctcagctgtcaccagcacttttgaccaagtcctgctactgcggcccctgccctagggcttagagcatggacctcctgccctgggggtcatctggggccagggctctctggatgccttcctgctgccccagccagggttggagtcttagcctcgggatccagtgaagccagaagccaaataaactcaaaagctgtctcccc

SEQ ID NO: 2 - protein, 847 aa, Homo sapien
MEPLKSLFLKSPLGSWNGSGSGGGGGGGGGRPEGSPKAAGYANPVWTALFDYEPSGQDELALRKGDRVEVLSRDAAISGDEGWWAGQVGGQVGIFPSNYVSRGGGPPPCEVASFQELRLEEVIGIGGFGKVYRGSWRGELVAVKAARQDPDEDISVTAESVRQEARLFAMLAHPNIIALKAVCLEEPNLCLVMEYAAGGPLSRALAGRRVPPHVLVNWAVQIARGMHYLHCEALVPVIHRDLKSNNILLLQPIESDDMEHKTLKITDFGLAREWHKTTQMSAAGTYAWMAPEVIKASTFSKGSDVWSFGVLLWELLTGEVPYRGIDCLAVAYGVAVNKLTLPIPSTCPEPFAQLMADCWAQDPHRRPDFASILQQLEALEAQVLREMPRDSFHSMQEGWKREIQGLFDELRAKEKELLSREEELTRAAREQRSQAEQLRRREHLLAQWELEVFERELTLLLQQVDRERPHVRRRRGTFKRSKLRARDGGERISMPLDFKHRITVQASPGLDRRRNVFEVGPGDSPTFPRFRAIQLEPAEPGQAWGRQSPRRLEDSSNGERRACWAWGPSSPKPGEAQNGRRRSRMDEATWYLDSDDSSPLGSPSTPPALNGNPPRPSLEPEEPKRPVPAERGSSSGTPKLIQRALLRGTALLASLGLGRDLQPPGGPGRERGESPTTPPTPTPAPCPTEPPPSPLICFSLKTPDSPPTPAPLLLDLGIPVGQRSAKSPRREEEPRGGTVSPPPGTSRSAPGTPGTPRSPPLGLISRPRPSPLRSRIDPWSFVSAGPRPSPLPSPQPAPRRAPWTLFPDSDPFWDSPPANPFQGGPQDCRAQTKDMGAQAPWVPEAGP

SEQ ID NO: 3 - DNA, 2447 bp, Homo sapiens
  CDS (70)...(1498)
tggagtgggagctcaagcaggattcttcccgagtccctggcatcctcagaagcttcaactctggaggcaatgggtcgaaaggaagaagatgactgcagttcctggaagaaacagaccaccaacatccggaaaaccttcatttttatggaagtgctgggatcaggagctttctcagaagttttcctggtgaagcaaagactgactgggaagctctttgctctgaagtgcatcaagaagtcacctgccttccgggacagcagcctggagaatgagattgctgtgttgaaaaagatcaagcatgaaaacattgtgaccctggaggacatctatgagagcaccacccactactacctggtcatgcagcttgtttctggtggggagctctttgaccggatcctggagcggggtgtctacacagagaaggatgccagtctggtgatccagcaggtcttgtcggcagtgaaatacctacatgagaatggcatcgtccacagagacttaaagcccgaaaacctgctttaccttacccctgaagagaactctaagatcatgatcactgactttggtctgtccaagatggaacagaatggcatcatgtccactgcctgtgggaccccaggctacgtggctccagaagtgctggcccagaaaccctacagcaaggctgtggattgctggtccatcggcgtcatcacctacatattgctctgtggataccccccgttctatgaagaaacggagtctaagcttttcgagaagatcaaggagggctactatgagtttgagtctccattctgggatgacatttctgagtcagccaaggactttatttgccacttgcttgagaaggatccgaacgagcggtacacctgtgagaaggccttgagtcatccctggattgacggaaacacggccctccaccgggacatctacccatcagtcagcctccagatccagaagaactttgctaagagcaagtggaggcaagccttcaacgcagcagctgtggtgcaccacatgaggaagctacacatgaacctgcacagcccgggcgtccgcccagaggtggagaacaggccgcctgaaactcaagcctcagaaacctctagacccagctcccctgagatcaccatcaccgaggcacctgtcctggaccacagtgtagcactccctgccctgacccaattaccctgccagcatggccgccggcccactgcccctggtggcaggtccctcaactgcctggtcaatggctccctccacatcagcagcagcctggtgcccatgcatcaggggtccctggccgccgggccctgtggctgctgctccagctgcctgaacattgggagcaaaggaaagtcctcctactgctctgagcccacactcctcaaaaaggccaacaaaaaacagaacttcaagtcggaggtcatggtaccagttaaagccagtggcagctcccactgccgggcagggcagactggagtctgtctcattatgtgattcctggagcctgtgcctatgtcactgcaattttcaggagacatattcaactcctctgctcttccaaacctggtgtctatccggcagagggaggaaggcagagcaagtggagcagggcttagcaggagcagtttctggccagaagcaccagcctgctgccagcggggcagcccctcataggaggcccaggagggagccccaaggcgtagaagccttgttgaagctgtgagcaggagaagcggtgcccaccagcttccaggtctccctgacctgcctgctctatgccccacaccctacgtgccgtggctctgtgcagtgtacgtagatagctctcgcctgggtctgtgctgtttgtcgtgaaaagcttaatgggctggccaggctgtgtcaccttctccaagcaaagccatatggagcatctacccagactcccactctgcacacactcactcccacctctcaagcctccaacctcttggccagattgggctcattaatgtcgttgcctgcccatctgcatgaatgacaggcagctccccatggtggtctgcctgtgagctcttcaagttctaatccttaactccaggattagctcccaagtgcgctgagacccagccagcacacttctggcccttctccctgcctcaatctaaaagcagtgccacaccctccaaagtggaatagaaagaagttcatgagtaagggctgcaaggaattcttatcctggccacatgtcctccgtgcacacacccaatggagttaaccttggaagttgactattttaatgtctgccaggagttctaatcctgcctctgttcccttttctctccttgaaagtccagcacaccattcttgtccttccccagtttcctcgccctccacccctccagcttcatgctcagtgttgtgcttaataaaatggacatatttttctctaaaaaaaaaaa

SEQ ID NO: 4 - protein, 476 aa, Homo sapiens [sequence omitted]

SEQ ID NO: 5 - DNA, 1812 bp, Homo sapiens
gaagagggcagagccgtgcatggggctgctccccaggacctgagcaggaacctggagttttcagagctgcctgatcattgctacagaatgaactctagcccagctgggaccccaagtccacagccctccagggccaatgggaacatcaacctggggccttcagccaacccaaatgcccagcccacggacttcgacttcctcaaagtcatcggcaaagggaactacgggaaggtcctactggccaagcgcaagtctgatggggcgttctatgcagtgaaggtactacagaaaaagtccatcttaaagaagaaagagcagagccacatcatggcagagcgcagtgtgcttctgaagaacgtgcggcaccccttcctcgtgggcctgcgctactccttccagacacctgagaagctctacttcgtgctcgactatgtcaacgggggagagctcttcttccacctgcagcgggagcgccggttcctggagccccgggccaggttctacgctgctgaggtggccagcgccattggctacctgcactccctcaacatcatttacagggatctgaaaccagagaacattctcttggactgccagggacacgtggtgctgacggattttggcctctgcaaggaaggtgtagagcctgaagacaccacatccacattctgtggtacccctgagtacttggcacctgaagtgcttcggaaagagccttatgatcgagcagtggactggtggtgcttgggggcagtcctctacgagatgctccatggcctgccgcccttctacagccaagatgtatcccagatgtatgagaacattctgcaccagccgctacagatccccggaggccggacagtggccgcctgtgacctcctgcaaagccttctccacaaggaccagaggcagcggctgggctccaaagcagactttcttgagattaagaaccatgtattcttcagccccataaactgggatgacctgtaccacaagaggctaactccacccttcaacccaaatgtgacaggacctgctgacttgaagcattttgacccagagttcacccaggaagctgtgtccaagtccattggctgtacccctgacactgtggccagcagctctggggcctcaagtgcattcctgggattttcttatgcgccagaggatgatgacatcttggattgctagaagagaaggacctgtgaaactactgaggccagctggtattagtaaggaattaccttcagctgctaggaagagcgactcaaactaacaatggcttcaacgagaagcaggtttattttttccagcacataaaagaaaaataatgtttcggagtccaggactggcaggacaggtcatcagatactcagaggctgtatctctgccctgccaaccttgacaaatggcttccaatgttaggtttgctacaagatggttactggagctctagctgcctattttgtgtttagggaagggaaaatggaggaaaggggagaagagcaaagggcgcttttaaagagctttcccaaaagctccccccaatgacttttgcttccatctcactaaccacccacccctacctggaatggaggctgggaaatgtggcttatttgctgggtacgtgactatccctaataacaaaggggttttgaccctaagacattaggggagaatgttgggtaggcagccagccctcttttaccatagggcctcctggtgtttggattttgatctcaatgtgtaaaatgacagagatgtaacaagctcatagggtatcaatatctcttattgttctatgttgaaaaa

SEQ ID NO: 6 - protein, 367 aa, Homo sapiens
MNSSPAGTPSPQPSRANGNINLGPSANPNAQPTDFDFLKVIGKGNYGKVLLAKRKSDGAFYAVKVLQKKSILKKKEQSHIMAERSVLLKNVRHPFLVGLRYSFQTPEKLYFVLDYVNGGELFFHLQRERRFLEPRARFYAAEVASAIGYLHSLNIIYRDLKPENILLDCQGHVVLTDFGLCKEGVEPEDTTSTFCGTPEYLAPEVLRKEPYDRAVDWWCLGAVLYEMLHGLPPFYSQDVSQMYENILHQPLQIPGGRTVAACDLLQSLLHKDQRQRLGSKADFLEIKNHVFFSPINWDDLYHKRLTPPFNPNVTGPADLKHFDPEFTQEAVSKSIGCTPDTVASSSGASSAFLGFSYAPEDDDILDC

SEQ ID NO: 7 - DNA, 2557 bp, Homo sapiens
cagagggaggaagaagcggcggcgcggcggcggcggctcctctttgcagagggggaaactcttgggctgagagcaggaacaacgcggtaggcaaggcgggctgctggctcccccggctccggcagcagcggcggcagcccgagcagcggcagcagcagcggcagcaccccaggcgctgacagccccgccggccggctccgttgctgaccgccgactgtcaatggagctggaaaacatcgtggccaacacggtcttgctgaaagccagggaagggggcggaggaaagcgcaaagggaaaagcaagaagtggaaagaaatcctgaagttccctcacattagccagtgtgaagacctccgaaggaccatagacagagattactgcagtttatgtgacaagcagccaatcgggaggctgcttttccggcagttttgtgaaaccaggcctgggctggagtgttacattcagttcctggactccgtggcagaatatgaagttactccagatgaaaaactgggagagaaagggaaggaaattatgaccaagtacctcaccccaaagtcccctgttttcatagcccaagttggccaagacctggtctcccagacggaggagaagctcctacagaagccgtgcaaagaactcttttctgcctgtgcacagtctgtccacgagtacctgaggggagaaccattccacgaatatctggacagcatgttttttgaccgctttctccagtggaagtggttggaaaggcaaccggtgaccaaaaacactttcaggcagtatcgagtgctaggaaaagggggcttcggggaggtctgtgcctgccaggttcgggccacgggtaaaatgtatgcctgcaagcgcttggagaagaagaggatcaaaaagaggaaaggggagtccatggccctcaatgagaagcagatcctcgagaaggtcaacagtcagtttgtggtcaacctggcctatgcctacgagaccaaggatgcactgtgcttggtcctgaccatcatgaatgggggtgacctgaagttccacatctacaacatgggcaaccctggcttcgaggaggagcgggccttgttttatgcggcagagatcctctgcggcttagaagacctccaccgtgagaacaccgtctaccgagatctgaaacctgaaaacatcctgttagatgattatggccacattaggatctcagacctgggcttggctgtgaagatccccgagggagacctgatccgcggccgggtgggcactgttggctacatggcccccgaagtcctgaacaaccagaggtacggcctgagccccgactactggggccttggctgcctcatctatgagatgatcgagggccagtcgccgttccgcggccgtaaggagaaggtgaagcgggaggaggtggaccgccgggtcctggagacggaggaggtgtactcccacaagttctccgaggaggccaagtccatctgcaagatgctgctcacgaaagatgcgaagcagaggctgggctgccaggaggagggggctgcagaggtcaagagacaccccttcttcaggaacatgaacttcaagcgcttagaagccgggatgttggaccctcccttcgttccagacccccgcgctgtgtactgtaaggacgtgctggacatcgagcagttctccactgtgaagggcgtcaatctggaccacacagacgacgacttctactccaagttctccacgggctctgtgtccatcccatggcaaaacgagatgatagaaacagaatgctttaaggagctgaacgtgtttggacctaatggtaccctcccgccagatctgaacagaaaccaccctccggaaccgcccaagaaagggctgctccagagactcttcaagcggcagcatcagaacaattccaagagttcgcccagctccaagaccagttttaaccaccacataaactcaaaccatgtcagctcgaactccaccggaagcagctagtttcggctctggcctccaagtccacagtggaaccagcccagacccttctccttagaagtggaagtagtggagcccctgctctggtggggctgccaggggagaccccgggagccggaaggaggccgtccatcccgtcgacgtagaacctcgaggtttctcaaagaaatttccactcaggtctgttttccgaggcggccccgggcgggtggattggatttgtctttggtgaacattgcaatagaaatccaattggatacgacaacttgcacgtattttaatagcgtcataactagaactgaattttgtctttatgatttttaaagaaaagttttgtaaatttctctactgtctcagtttacattttcggtatatttgtatttaaatgaagtgagactttgagggtgtatattttctgtgcagccactgttaagccatgtgttccaaggcattttagcggggagggggttatcaaaaaaaaaaaaaatgtgactcaagacttccagagcctcaaatgagaaaatgtctttattaaatgtagaaagtgatccatacttcaaaaaaaaa

SEQ ID NO: 8 - protein, 590 aa, Homo sapiens
MELENIVANTVLLKAREGGGGKRKGKSKKWKEILKFPHISQCEDLRRTIDRDYCSLCDKQPIGRLLFRQFCETRPGLECYIQFLDSVAEYEVTPDEKLGEKGKEIMTKYLTPKSPVFIAQVGQDLVSQTEEKLLQKPCKELFSACAQSVHEYLRGEPFHEYLDSMFFDRFLQWKWLERQPVTKNTFRQYRVLGKGGFGEVCACQVRATGKMYACKRLEKKRIKKRKGESMALNEKQILEKVNSQFVVNLAYAYETKDALCLVLTIMNGGDLKFHIYNMGNPGFEEERALFYAAEILCGLEDLHRENTVYRDLKPENILLDDYGHIRISDLGLAVKIPEGDLIRGRVGTVGYMAPEVLNNQRYGLSPDYWGLGCLIYEMIEGQSPFRGRKEKVKREEVDRRVLETEEVYSHKFSEEAKSICKMLLTKDAKQRLGCQEEGAAEVKRHPFFRNMNFKRLEAGMLDPPFVPDPRAVYCKDVLDIEQFSTVKGVNLDHTDDDFYSKFSTGSVSIPWQNEMIETECFKELNVFGPNGTLPPDLNRNHPPEPPKKGLLQRLFKRQHQNNSKSSPSSKTSFNHHINSNHVSSNSTGSS

SEQ ID NO: 9 - DNA, 3407 bp, Homo sapiens
cagggagggcttggctccaccactttcctcccccagcctttgggcagcaggtcacccctgttcaggctctgagggtgccccctcctggtcctgtcctcaccaccccttccccacctcctgggaaaaaaaaaaaaaaaaaaaaaaaagctggtttaaagcagagagcctgagggctaaatttaactgtccgagtcggaatccatctctgagtcacccaagaagctgccctggcctcccgtccccttcccaggcctcaacccctttctcccacccagccccaacccccagccctcaccccctagcccccagttctggagcttgtcgggagcaagggggtggttgctactgggtcactcagcctcaattggccctgttcagcaatgggcaggttcttcttgaaattcatcacacctgtggcttcctctgtgctctacctttttattggggtgacagtgtgacagctgagattctccatgcattccccctactctagcactgaagggttctgaagggccctggaaggagggagcttggggggctggcttgtgaggggttaaggctgggaggcgggaggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggagacactgtcggacattcgggaaggtgcgccgctaggggtccacctgccttttgtgggctactcctactcctgcatggccctcagggacagtgaggtcccaggccccacacccatggaagtggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgtaccgggcccgccccctagcggccggggagggaggggccgggtccgcggccggcgaacggggctcgaagggtccttgtagccgggaatgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgccc

SEQ ID NO: 10 - protein, 629 aa, Homo sapiens [sequence omitted]

SEQ ID NO: 11 - DNA, 2637 bp, Homo sapiens
  CDS (1)...(2637)
atggccaccgccccctcttatcccgccgggctccctggctctcccgggccggggtctcctccgccccccggcggcctagagctgcagtcgccgccaccgctactgccccagatcccggccccgggttccggggtctcctttcacatccagatcgggctgacccgcgagttcgtgctgttgcccgccgcctccgagctggctcatgtgaagcagctggcctgttccatcgtggaccagaagttccctgagtgtggcttctacggcctttacgacaagatcctgcttttcaaacatgaccccacgtcggccaacctcctgcagctggtgcgctcgtccggagacatccaggagggcgacctggtggaggtggtgctgtcggcctcggccaccttcgaggacttccagatccgcccgcacgccctcacggtgcactcctatcgggcgcctgccttctgtgatcactgcggggagatgctcttcggcctagtgcgccagggcctcaagtgcgatggctgcgggctgaactaccacaagcgctgtgccttcagcatccccaacaactgtagtggggcccgcaaacggcgcctgtcatccacgtctctggccagtggccactcggtgcgcctcggcacctccgagtccctgccctgcacggctgaagagctgagccgtagcaccaccgaactcctgcctcgccgtcccccgtcatcctcttcctcctcttctgcctcatcgtatacgggccgccccattgagctggacaagatgctgctctccaaggtcaaggtgccgcacaccttcctcatccacagctatacacggcccaccgtttgccaggcttgcaagaaactcctcaagggcctcttccggcagggcctgcaatgcaaagactgcaagtttaactgtcacaaacgctgcgccacccgcgtccctaatgactgcctgggggaggcccttatcaatggagatgtgccgatggaggaggccaccgatttcagcgaggctgacaagagcgccctcatggatgagtcagaggactccggtgtcatccctggctcccactcagagaatgcgctccacgccagtgaggaggaggaaggcgagggaggcaaggcccagagctccctggggtacatccccctaatgagggtggtgcaatcggtgcgacacacgacgcggaaatccagcaccacgctgcgggagggttgggtggttcattacagcaacaaggacacgctgagaaagcggcactattggcgcctggactgcaagtgtatcacgctcttccagaacaacacgaccaacagatactataaggaaattccgctgtcagaaatcctcacggtggagtccgcccagaacttcagccttgtgccgccgggcaccaacccacactgctttgagatcgtcactgccaatgccacctacttcgtgggcgagatgcctggcgggactccgggtgggccaagtgggcagggggctgaggccgcccggggctgggagacagccatccgccaggccctgatgcccgtcatccttcaggacgcacccagcgccccaggccacgcgccccacagacaagcttctctgagcatctctgtgtccaacagtcagatccaagagaatgtggacattgccactgtctaccagatcttccctgacgaagtgctgggctcagggcagtttggagtggtctatggagggaaacaccggaagacaggccgggacgtggcagttaaggtcattgacaaactgcgcttccctaccaagcaggagagccagctccggaatgaagtggccattctgcagagcctgcggcatcccgggatcgtgaacctggagtgcatgttcgagacgcctgagaaagtgtttgtggtgatggagaagctgcatggggacatgttggagatgatcctgtccagtgagaagggccggctgcctgagcgcctcaccaagttcctcatcacccagatcctggtggctttgagacaccttcacttcaagaacattgtccactgtgacttgaaaccagaaaacgtgttgctggcatcagcagacccatttcctcaggtgaagctgtgtgactttggctttgctcgcatcatcggcgagaagtcgttccgccgctcagtggtgggcacgccggcctacctggcacccgaggtgctgctcaaccagggctacaaccgctcgctggacatgtggtcagtgggcgtgatcatgtacgtcagcctcagcggcaccttccctttcaacgaggatgaggacatcaatgaccagatccagaacgccgccttcatgtacccggccagcccctggagccacatctcagctggagccattgacctcatcaacaacctgctgcaggtgaagatgcgcaaacgctacagcgtggacaaatctctcagccacccctggttacaggagtaccagacgtggctggacctccgagagctggaggggaagatgggagagcgatacatcacgcatgagagtgacgacgcgcgctgggagcagtttgcagcagagcatccgctgcctgggtctgggctgcccacggacagggatctcggtggggcctgtccaccacaggaccacgacatgcaggggctggcggagcgcatcagtgttctctga

SEQ ID NO: 12 - protein, 878 aa, Homo sapiens
MATAPSYPAGLPGSPGPGSPPPPGGLELQSPPPLLPQIPAPGSGVSFHIQIGLTREFVLLPAASELAHVKQLACSIVDQKFPECGFYGLYDKILLFKHDPTSANLLQLVRSSGDIQEGDLVEVVLSASATFEDFQIRPHALTVHSYRAPAFCDHCGEMLFGLVRQGLKCDGCGLNYHKRCAFSIPNNCSGARKRRLSSTSLASGHSVRLGTSESLPCTAEELSRSTTELLPRRPPSSSSSSSASSYTGRPIELDKMLLSKVKVPHTFLIHSYTRPTVCQACKKLLKGLFRQGLQCKDCKFNCHKRCATRVPNDCLGEALINGDVPMEEATDFSEADKSALMDESEDSGVIPGSHSENALHASEEEEGEGGKAQSSLGYIPLMRVVQSVRHTTRKSSTTLREGWVVHYSNKDTLRKRHYWRLDCKCITLFQNNTTNRYYKEIPLSEILTVESAQNFSLVPPGTNPHCFEIVTANATYFVGEMPGGTPGGPSGQGAEAARGWETAIRQALMPVILQDAPSAPGHAPHRQASLSISVSNSQIQENVDIATVYQIFPDEVLGSGQFGVVYGGKHRKTGRDVAVKVIDKLRFPTKQESQLRNEVAILQSLRHPGIVNLECMFETPEKVFVVMEKLHGDMLEMILSSEKGRLPERLTKFLITQILVALRHLHFKNIVHCDLKPENVLLASADPFPQVKLCDFGFARIIGEKSFRRSVVGTPAYLAPEVLLNQGYNRSLDMWSVGVIMYVSLSGTFPFNEDEDINDQIQNAAFMYPASPWSHISAGAIDLINNLLQVKMRKRYSVDKSLSHPWLQEYQTWLDLRELEGKMGERYITHESDDARWEQFAAEHPLPGSGLPTDRDLGGACPPQDHDMQGLAERISVL

SEQ ID NO: 13 - DNA, 2037 bp, Homo sapiens
  CDS (1)...(2037)
atggccaccgccccctcttatcccgccgggctccctggctctcccgggccggggtctcctccgccccccggcggcctagagctgcagtcgccgccaccgctactgccccagatcccggccccgggttccggggtctcctttcacatccagatcgggctgacccgcgagttcgtgctgttgcccgccgcctccgagctggctcatgtgaagcagctggcctgttccatcgtggaccagaagttccctgagtgtggcttctacggcctttacgacaagatcctgcttttcaaacatgaccccacgtcggccaacctcctgcagctggtgcgctcgtccggagacatccaggagggcgacctggtggaggtggtgctgtcggcctcggccaccttcgaggacttccagatccgcccgcacgccctcacggtgcactcctatcgggcgcctgccttctgtgatcactgcggggagatgctcttcggcctagtgcgccagggcctcaagtgcgatggctgcgggctgaactaccacaagcgctgtgccttcagcatccccaacaactgtagtggggcccgcaaacggcgcctgtcatccacgtctctggccagtggccactcggtgcgcctcggcacctccgagtccctgccctgcacggctgaagagctgagccgtagcaccaccgaactcctgcctcgccgtcccccgtcatcctcttcctcctcttctgcctcatcgtatacgggccgccccattgagctggacaagatgctgctctccaaggtcaaggtgccgcacaccttcctcatccacagctatacacggcccaccgtttgccaggcttgcaagaaactcctcaagggcctcttccggcagggcctgcaatgcaaagactgcaagtttaactgtcacaaacgctgcgccacccgcgtccctaatgactgcctgggggaggcccttatcaatggagatgtgccgatggaggaggccaccgatttcagcgaggctgacaagagcgccctcatggatgagtcagaggactccggtgtcatccctggctcccactcagagaatgcgctccacgccagtgaggaggaggaaggcgagggaggcaaggcccagagctccctggggtacatccccctaatgagggtggtgcaatcggtgcgacacacgacgcggaaatccagcaccacgctgcgggagggttgggtggttcattacagcaacaaggacacgctgagaaagcggcactattggcgcctggactgcaagtgtatcacgctcttccagaacaacacgaccaacagatactataaggaaattccgctgtcagaaatcctcacggtggagtccgcccagaacttcagccttgtgccgccgggcaccaacccacactgctttgagatcgtcactgccaatgccacctacttcgtgggcgagatgcctggcgggactccgggtgggccaagtgggcagggggctgaggccgcccggggctgggagacagccatccgccaggccctgatgcccgtcatccttcaggacgcacccagcgccccaggccacgcgccccacagacaagcttctctgagcatctctgtgtccaacagtcagatccaagagaatgtggacattgccactgtctaccagatcttccctgacgaagtgctgggctcagggcagtttggagtggtctatggagggaaacaccggaagacaggccgggacgtggcagttaaggtcattgacaaactgcgcttccctaccaagcaggagagccagctccggaatgaagtggccattctgcagagcctgcggcatcccgggatcgtgaacctggagtgcatgttcgagacgcctgagaaagtgtttgtggtgatggagaagctgcatggggacatgttggagatgatcctgtccagtgagaagggccggctgcctgagcgcctcaccaagttcctcatcacccagatttctgctttctgggctcttgcctgccccacacctaagccctgtgctaagccctttacctcctga

SEQ ID NO: 14 - protein, 678 aa, Homo sapiens
MATAPSYPAGLPGSPGPGSPPPPGGLELQSPPPLLPQIPAPGSGVSFHIQIGLTREFVLLPAASELAHVKQLACSIVDQKFPECGFYGLYDKILLFKHDPTSANLLQLVRSSGDIQEGDLVEVVLSASATFEDFQIRPHALTVHSYRAPAFCDHCGEMLFGLVRQGLKCDGCGLNYHKRCAFSIPNNCSGARKRRLSSTSLASGHSVRLGTSESLPCTAEELSRSTTELLPRRPPSSSSSSSASSYTGRPIELDKMLLSKVKVPHTFLIHSYTRPTVCQACKKLLKGLFRQGLQCKDCKFNCHKRCATRVPNDCLGEALINGDVPMEEATDFSEADKSALMDESEDSGVIPGSHSENALHASEEEEGEGGKAQSSLGYIPLMRVVQSVRHTTRKSSTTLREGWVVHYSNKDTLRKRHYWRLDCKCITLFQNNTTNRYYKEIPLSEILTVESAQNFSLVPPGTNPHCFEIVTANATYFVGEMPGGTPGGPSGQGAEAARGWETAIRQALMPVILQDAPSAPGHAPHRQASLSISVSNSQIQENVDIATVYQIFPDEVLGSGQFGVVYGGKHRKTGRDVAVKVIDKLRFPTKQESQLRNEVAILQSLRHPGIVNLECMFETPEKVFVVMEKLHGDMLEMILSSEKGRLPERLTKFLITQISAFWALACPTPKPCAKPFTS

SEQ ID NO: 15 - DNA, 28 bp, Primer sequence 1
gaattccatggagcccttgaagagcctc

SEQ ID NO: 16 - DNA, 28 bp, Primer sequence 2
ctcgagtcaaggccccgcttccggcacc

SEQ ID NO: 17 - DNA, 23 bp, Homo sapiens
ggagggcgaggaaactggggaag

SEQ ID NO: 18 - DNA, 30 bp, Homo sapiens
ggatccatgaactctagcccagctgggacc

SEQ ID NO: 19 - DNA, 30 bp, Homo sapiens
gaattctagcaatccaagatgtcatcatcc

SEQ ID NO: 20 - DNA, 30 bp, Homo sapiens
ggatccatggagctggaaaacatcgtggcc

SEQ ID NO: 21 - DNA, 28 bp, Homo sapiens
gaattctagctgcttccggtggagttcg

SEQ ID NO: 22 - DNA, 28 bp, Homo sapiens
gaattccatgtcagccgaggtgcggctg

SEQ ID NO: 23 - DNA, 28 bp, Homo sapiens
gcggccgctcagggagcgcgggcggctc

SEQ ID NO: 24 - DNA, 25 bp, Homo sapiens
gtggagggcgaggaaactggggaag

SEQ ID NO: 25 - DNA, 31 bp, Homo sapiens
ctcgagtcacataatgagacagactccagtc

SEQ ID NO: 26 - protein, 13 aa, Homo sapiens
KRREILSRRPSYR

SEQ ID NO: 27 - protein, 15 aa, Homo sapiens
PLARTLSVAGLPGKK

SEQ ID NO: 28 - protein, 10 aa, Homo sapiens
PLSRTLSVSS

SEQ ID NO: 29 - DNA, 30 bp, Homo sapiens
gaattcaatgggtcgaaaggaagaagatga

SEQ ID NO: 30 - DNA, 30 bp, Homo sapiens; identical to SEQ ID NO: 29

SEQ ID NO: 31 - DNA, 30 bp, Homo sapiens
ctcgagctggatctggaggctgactgatgg

SEQ ID NO: 32 - protein, 11 aa, Homo sapiens
CKRPRAASFAE

SEQ ID NO: 33 - protein, 35 aa, Homo sapiens
KTFCGTPEYLAPEVRREPRILSEEEQEMFRDFDYI

SEQ ID NO: 34 - protein, 11 aa, Homo sapiens
CKRPRAASFAE

SEQ ID NO: 35 - protein, 35 aa, Homo sapiens; identical to SEQ ID NO: 33

SEQ ID NO: 36 - protein, 11 aa, Homo sapiens
CKRPRAASFAE

SEQ ID NO: 37 - protein, 10 aa, Homo sapiens
CGRTGRRNSI

SEQ ID NO: 38 - protein, 530 aa, Homo sapiens
MSAEVRLRRLQQLVLDPGFLGLEPLLDLLLGVHQELGASELAQDKYVADFLQWAEPIVVRLKEVRLQRDDFEILKVIGRGAFSEVAVVKMKQTGQVYAMKIMNKWDMLKRGEVSCFREERDVLVNGDRRWITQLHFAFQDENYLYLVMEYYVGGDLLTLLSKFGERIPAEMARFYLAEIVMAIDSVHRLGYVHRDIKPDNILLDRCGHIRLADFGSCLKLRADGTVRSLVAVGTPDYLSPEILQAVGGGPGTGSYGPECDWWALGVFAYEMFYGQTPFYADSTAETYGKIVHYKEHLSLPLVDEGVPEEARDFIQRSLCPPETRLGRGGAGDFRTHPFFFGLDWDGLRDSVPPFTPDFEGATDTCNFDLVEDGLTAMETLSDIREGAPLGVHLPFVGYSYSCMALRDSEVPGPTPMELEAEQLLEPHVQAPSLEPSVSPQDETAEVAVPAAVPAAEAEAEVTLRELQEALEEEVLTRQSLSREMEAIRTDNQNFASQLREAEARNRDLEAHVRQLQERMELLQAEGATGP

SEQ ID NO: 39 - protein, 599 aa, Homo sapiens
MSAEVRLRRLQQLVLDPGFLGLEPLLDLLLGVHQELGASELAQDKYVADFLQWAEPIVVRLKEVRLQRDDFEILKVIGRGAFSEVAVVKMKQTGQVYAMKIMNKWDMLKRGEVSCFREERDVLVNGDRRWITQLHFAFQDENYLYLVMEYYVGGDLLTLLSKFGERIPAEMARFYLAEIVMAIDSVHRLGYVHRDIKPDNILLDRCGHIRLADFGSCLKLRADGTVRSLVAVGTPDYLSPEILQAVGGGPGTGSYGPECDWWALGVFAYEMFYGQTPFYADSTAETYGKIVHYKEHLSLPLVDEGVPEEARDFIQRLLCPPETRLGRGGAGDFRTHPFFFGLDWDGLRDSVPPFTPDFEGATDTCNFDLVEDGLTAMVSGGGETLSDIREGAPLGVHLPFVGYSYSCMALRDSEVPGPTPMEVEAEQLLEPHVQAPSLEPSVSPQDETAEVAVPAAVPAAEAEAEVTLRELQEALEEEVLTRQSLSREMEAIRTDNQNFASQLREAEARNRDLEAHVRQLQERMELLQAEGATAVTGVPSPRATDPPSHVPRPGLSEALSLLLFAVVLSRAAALGCIGLVAHAGQLTAVWRRPGAARAP